(19) United States
(12) Patent Application Publication
(10) Pub. No.: US 2021/0269517 A1
(43) Pub. Date: Sep. 2, 2021

(54) METHOD OF TREATING WASTING DISORDERS

(71) Applicants: CSL Limited, Parkville, Victoria (AU); CSL Innovation Pty Ltd, Parkville, Victoria (AU); B-Creative Sweden AB, Balsta (SE)

(72) Inventors: Ulf ERIKSSON, Balsta (SE); Annelie FALKEVALL, Balsta (SE); Annika MEHLEM, Balsta (SE); Isolde PALOMBO, Balsta (SE); Samuel WRIGHT, Parkville, Victoria (AU)

(73) Assignees: CSL Limited, Parkville, Victoria (AU); CSL Innovation Pty Ltd, Parkville, Victoria (AU); B-Creative Sweden AB, Balsta (AU)

(21) Appl. No.: 17/276,611

(22) PCT Filed: Sep. 18, 2019
(86) PCT No.: PCT/AU2019/050998

§ 371 Date: Mar. 16, 2021

Related U.S. Application Data
(60) Provisional application No. 62/732,727, filed on Sep. 18, 2018.

(51) Int. Cl. C07K 16/22; C12N 15/113; A61P 3/00

(52) U.S. Cl. CPC C07K 16/22; C12N 15/1136; A61P 3/00; C07K 2317/51; C07K 2317/515; C07K 2317/54; C07K 2317/565; C07K 2317/622; C07K 2317/626; C12N 2310/14; C12N 2310/127; C12N 2310/12; C07K 2317/55

(57) ABSTRACT

The present disclosure provides a method of treating a wasting disorder in a subject, the method comprising administering to the subject a compound that inhibits VEGF-B signalling. The present disclosure also provides a method of treating cancer cachexia in a subject suffering from cancer cachexia, the method comprising 5 administering to the subject a compound that inhibits VEGF-B signaling.

### RELATED APPLICATION DATA

The present application claims priority from U.S. Application No. 62/732,727 entitled “Method of Treating Wasting Disorders” filed on 18 Sep. 2018. The entire contents of which is hereby incorporated by reference.

### SEQUENCE INFORMATION

The present application is filed with a Sequence Listing in electronic form. The entire contents of the Sequence Listing are hereby incorporated by reference.

### FIELD

The present disclosure relates to a method of treating a wasting disorder in a subject by antagonizing vascular endothelial growth factor (VEGF)-B.

### BACKGROUND

Wasting disorders, or wasting syndrome, refers to the progressive loss of weight and/or muscle mass and the progressive weakening and degeneration of muscle in a subject. Wasting disorders are class of disorders that include cachexia, anorexia, muscle wasting and/or fat wasting.

Cachexia is a complex metabolic wasting disorder that is characterized by loss of body weight and loss of muscle and fat mass. Cachexia is distinct from starvation, malabsorption and hyperthyroidism and is associated with increased morbidity. Cachexia commonly occurs in subjects suffering from chronic illness, such as cancer (i.e., cancer cachexia), with up to 80% of all cancer patients eventually developing cachexia. Cachexia is a debilitating disorder and is associated with reduced mobility, with individuals suffering from cachexia having increased risk of complications in surgery, impaired response to chemo-/radio-therapy, decreased survival time and increased psychological distress, leading to an overall reduction in quality of life.

Current therapies for cachexia include medication aimed at retarding or halting progression of the disorder. Treatments include orexigenic agents (i.e., appetite stimulants), corticosteroids, cannabinoids, serotonin antagonists, prokinetic agents, androgens and anabolic agents, anticytokine agents, non-steroidal anti-inflammatory drugs, and regulators of circadian rhythm. Most therapies are directed to treating the underlying or associated condition (e.g., cancer). However, often such treatment is compromised by the patients' inability to tolerate such treatments due to their cachexia. Thus, there is a need in the art for improved treatments of wasting disorders, such as cachexia.

### SUMMARY

In producing the present invention, the inventors studied the effects of inhibiting signaling of VEGF-B in a mouse model of fasting-induced lipolysis. The inventors studied the effect of this growth factor by preventing expression of VEGF-B (e.g., using genetically-modified mice in which expression of VEGF-B is reduced or prevented) or by administering an antagonist of VEGF-B (e.g., an antagonist antibody). The present inventors have found that inhibition of VEGF-B signaling resulted in a reduction in basal lipolysis rate. The inventors also found that antagonism of VEGF-B signaling decreased or prevented an increase in levels of plasma non-esterified fatty acids and free glycerol and hepatic lipid accumulation, as well as an increase in the expression of hepatic fatty acid transporters. The inventors found that the changes in basal lipolysis rate occurred in the absence of an effect on blood glucose levels indicating that inhibiting VEGF-B provides a benefit through a pathway additional to or other than glycemic control.

The findings by the inventors provide the basis for methods of inhibiting or downregulating lipolysis in a subject in need thereof by inhibiting VEGF-B signalling. For example, the present disclosure provides a method for inhibiting lipolysis in a subject in need thereof, the method comprising administering to the subject a compound that inhibits VEGF-B signaling.

In one example, the subject is in need of reduced lipolysis. For example, the subject has or is suffering from elevated lipolysis.

The findings by the inventors also provide the basis for methods for treating a wasting disorder in a subject by inhibiting VEGF-B signaling. For example, the present disclosure provides a method of treating a wasting disorder in a subject, the method comprising administering to the subject a compound that inhibits VEGF-B signaling.

In one example, the subject is suffering from a wasting disorder (i.e., the subject is in need of treatment).

In one example, the wasting disorder is selected from the group consisting of cachexia, unintended body weight loss, fat wasting.

In one example, the wasting disorder is cachexia. For example, the cachexia is pre-cachexia. In another example, the cachexia is overt cachexia. In a further example, the cachexia is refractory cachexia.

In one example, the subject is suffering from cachexia (i.e., the subject is in need of treatment).

In one example, the cachexia is selected from the group consisting of cancer cachexia, chronic kidney disease cachexia and diabetic cachexia.

In one example, the cachexia is cancer cachexia (i.e., the subject has or is suffering from cancer). For example, the cancer includes, but is not limited to, solid tumors, carcinoma, neuroma, melanoma, leukemia, lymphoma, sarcoma, fibroma, thyroid cancer, bladder cancer, lung cancer, blastoma, bone cancer, bone tumor, brain stem glioma, brain tumor, breast cancer, bronchial tumor, cervical cancer, colon cancer, colorectal cancer, neuroepithelial tumor, endometrial cancer, endometrial uterine cancer, fallopian tube cancer, kidney cancer, liver cancer, oral cancer, myeloma, neoplasm, neurinoma, neuroblastoma, ovarian cancer, pancreatic cancer, prostate cancer, rectal cancer and renal cell carcinoma.

In one example, the cachexia is chronic kidney disease cachexia (i.e., the subject has or is suffering from chronic kidney disease).

In one example, the cachexia is diabetic cachexia (i.e., the subject has or is suffering from diabetes). For example, the cachexia is associated with or caused by type 1 diabetes. In another example, the cachexia is associated with or caused by type 2 diabetes.

In one example, the wasting disorder is unintended body weight loss.

In one example, the wasting disorder is fat wasting.

In one example, the wasting disorder is anorexia.

The present disclosure additionally provides a method of treating cancer cachexia in a subject suffering from cancer cachexia, the method comprising administering to the subject a compound that inhibits VEGF-B signaling.

In one example, the compound is administered in an amount effective to have one or more of the following effects:

Reduce or prevent lipolysis;

Reduce or prevent hepatic lipid accumulation;

Reduce or prevent an increase in plasma non-esterified fatty acid levels; and/or

Reduce or prevent an increase in plasma free glycerol levels.

In one example, the compound that inhibits VEGF-B signaling specifically inhibits VEGF-B signaling. This does not mean that a method of the present disclosure does not encompass inhibiting signaling of multiple VEGF proteins, only that the compound (or part thereof) that inhibits VEGF-B signaling is specific to VEGF-B, e.g., is not a general inhibitor of VEGF proteins. This term also does not exclude, e.g., a bispecific antibody or protein comprising binding domains thereof, which can specifically inhibit VEGF-B signaling with one (or more) binding domains and can specifically inhibit signaling of another protein with another binding domain.

In one example, a compound that inhibits VEGF-B signaling binds to VEGF-B. For example, the compound is a protein comprising an antibody variable region that binds to or specifically binds to VEGF-B and neutralizes VEGF-B signaling.

In one example, the compound is an antibody mimetic. For example, the compound is a protein comprising an antigen binding domain of an immunoglobulin, e.g., an IgNAR, a camelid antibody or a T cell receptor.

In one example, a compound is a domain antibody (e.g., comprising only a heavy chain variable region or only a light chain variable region that binds to VEGF-B) or a heavy chain only antibody (e.g., a camelid antibody or an IgNAR) or variable region thereof.

In one example, a compound is a protein comprising a Fv. For example, the protein is selected from the group consisting of:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)₂;
(viii) a Fv; or
(ix) one of (i) to (viii) linked to a constant region of an antibody, Fc or a heavy chain constant domain (C_{H})2 and/or C_{H}3.

In another example, a compound is an antibody. Exemplary antibodies are full-length and/or naked antibodies.

In one example, the compound is a protein that is recombinant, chimeric, CDR grafted, humanized, synhumanized, primatized, deimmunized or human.

In one example, the compound is a protein comprising an antibody variable region that competitively inhibits the binding of antibody 2H10 to VEGF-B. In one example, the protein comprises a heavy chain variable region (V_{H}) comprising a sequence set forth in SEQ ID NO: 3 and a light chain variable region (V_{L}) comprising a sequence set forth in SEQ ID NO: 4.

In one example, the compound is a protein comprising a humanized variable region of antibody 2H10. For example, the protein comprises a variable region comprising the complementarity determining regions (CDRs) of the V_{H} and/or the V_{L} of antibody 2H10. For example, the protein comprises:

(i) a V_{H} comprising:
(a) a CDR1 comprising a sequence set forth in amino acids 25-34 of SEQ ID NO: 3;
(b) a CDR2 comprising a sequence set forth in amino acids 49-65 of SEQ ID NO: 3; and
(c) a CDR3 comprising a sequence set forth in amino acids 98-108 of SEQ ID NO: 3; and/or
   (ii) a V_{L} comprising:
(a) a CDR1 comprising a sequence set forth in amino acids 23-33 of SEQ ID NO: 4;
(b) a CDR2 comprising a sequence set forth in amino acids 49-55 of SEQ ID NO: 4; and
(c) a CDR3 comprising a sequence set forth in amino acids 88-96 of SEQ ID NO: 4.

In one example, the compound is a protein comprising a V_{H} and a V_{L}, the V_{H} and V_{L} being humanized variable regions of antibody 2H10. For example, the protein comprises:

(i) a V_{H} comprising:
(a) a CDR1 comprising a sequence set forth in amino acids 25-34 of SEQ ID NO: 3;
(b) a CDR2 comprising a sequence set forth in amino acids 49-65 of SEQ ID NO: 3; and
(c) a CDR3 comprising a sequence set forth in amino acids 98-108 of SEQ ID NO: 3; and
   (ii) a V_{L} comprising:
(a) a CDR1 comprising a sequence set forth in amino acids 23-33 of SEQ ID NO: 4;
(b) a CDR2 comprising a sequence set forth in amino acids 49-55 of SEQ ID NO: 4; and
(c) a CDR3 comprising a sequence set forth in amino acids 88-96 of SEQ ID NO: 4.

In one example, the variable region or V_{H} in any of the foregoing paragraphs comprises a sequence set forth in SEQ ID NO: 5.

In one example, the variable region or V_{L} in any of the foregoing paragraphs comprises a sequence set forth in SEQ ID NO: 6.

In one example, the compound is an antibody.

In one example, the compound is an antibody comprising a V_{H} comprising a sequence set forth in SEQ ID NO: 5 and a V_{L} comprising a sequence set forth in SEQ ID NO: 6.

In one example, the protein or antibody is any form of the protein or antibody encoded by a nucleic acid encoding any of the foregoing proteins or antibodies.

In one example, the protein or antibody comprises:

(i) a V_{H} comprising:
(a) a CDR1 comprising a sequence encoded by a nucleic acid comprising SEQ ID NO: 14 or comprising an amino acid sequence of SEQ ID NO: 20;
(b) a CDR2 comprising a sequence encoded by a nucleic acid comprising SEQ ID NO: 15 or comprising an amino acid sequence of SEQ ID NO: 21; and
(c) a CDR3 comprising a sequence encoded by a nucleic acid comprising SEQ ID NO: 16 or comprising an amino acid sequence of SEQ ID NO: 22; and/or
   (ii) a V_{L} comprising:
(a) a CDR1 comprising a sequence encoded by a nucleic acid comprising SEQ ID NO: 11 or comprising an amino acid sequence of SEQ ID NO: 17;
(b) a CDR2 comprising a sequence encoded by a nucleic acid comprising SEQ ID NO: 12 or comprising an amino acid sequence of SEQ ID NO: 18; and
(c) a CDR3 comprising a sequence encoded by a nucleic acid comprising SEQ ID NO: 13 or comprising an amino acid sequence of SEQ ID NO: 19.

In one example, the protein or antibody comprises:

(i) a V_{H} comprising:
(a) a CDR1 comprising a sequence encoded by a nucleic acid comprising SEQ ID NO: 26 or comprising an amino acid sequence of SEQ ID NO: 32;
(b) a CDR2 comprising a sequence encoded by a nucleic acid comprising SEQ ID NO: 27 or comprising an amino acid sequence of SEQ ID NO: 33; and
(c) a CDR3 comprising a sequence encoded by a nucleic acid comprising SEQ ID NO: 28 or comprising an amino acid sequence of SEQ ID NO: 34; and/or
   (ii) a V_{L} comprising:
(a) a CDR1 comprising a sequence encoded by a nucleic acid comprising SEQ ID NO: 23 or comprising an amino acid sequence of SEQ ID NO: 29;
(b) a CDR2 comprising a sequence encoded by a nucleic acid comprising SEQ ID NO: 24 or comprising an amino acid sequence of SEQ ID NO: 30; and
(c) a CDR3 comprising a sequence encoded by a nucleic acid comprising SEQ ID NO: 25 or comprising an amino acid sequence of SEQ ID NO: 31.

In one example, the protein or antibody comprises:

(i) a V_{H} comprising:
(a) a CDR1 comprising a sequence encoded by a nucleic acid comprising SEQ ID NO: 38 or comprising an amino acid sequence of SEQ ID NO: 44;
(b) a CDR2 comprising a sequence encoded by a nucleic acid comprising SEQ ID NO: 39 or comprising an amino acid sequence of SEQ ID NO: 45; and
(c) a CDR3 comprising a sequence encoded by a nucleic acid comprising SEQ ID NO: 40 or comprising an amino acid sequence of SEQ ID NO: 46; and/or
   (ii) a V_{L} comprising:
(a) a CDR1 comprising a sequence encoded by a nucleic acid comprising SEQ ID NO: 35 or comprising an amino acid sequence of SEQ ID NO: 41;
(b) a CDR2 comprising a sequence encoded by a nucleic acid comprising SEQ ID NO: 36 or comprising an amino acid sequence of SEQ ID NO: 42; and
(c) a CDR3 comprising a sequence encoded by a nucleic acid comprising SEQ ID NO: 37 or comprising an amino acid sequence of SEQ ID NO: 43.

In one example, the compound is within a composition. For example, the composition comprises a protein comprising an antibody variable region or a V_{H} or a V_{L} or an antibody as described herein. In one example, the composition additionally comprises one or more variants of the protein or antibody. For example, that comprises a variant missing an encoded C-terminal lysine residue, a deamidated variant and/or a glycosylated variant and/or a variant comprising a pyroglutamate, e.g., at the N-terminus of a protein and/or a variant lacking a N-terminal residue, e.g., a N-terminal glutamine in an antibody or V region and/or a variant comprising all or part of a secretion signal. Deamidated variants of encoded asparigine residues may result in isoaspartic, and aspartic acid isoforms being generated or even a succinamide involving an adjacent amino acid residue. Deamidated variants of encoded glutamine residues may result in glutamic acid. Compositions comprising a heterogeneous mixture of such sequences and variants are intended to be included when reference is made to a particular amino acid sequence.

In one example, the compound is a nucleic acid that inhibits VEGF-B signaling inhibits or prevents expression of VEGF-B. For example, the nucleic acid is selected from the group an antisense, a siRNA, a RNAi, a ribozyme and a DNAzyme.

In one example, the VEGF-B is mammalian VEGF-B, e.g., human VEGF-B.

In one example, the subject is a mammal, for example a primate, such as a human.

Methods of treatment described herein can additionally comprise administering a further treatment for a wasting disorder (e.g., cachexia).

Methods of treatment of a wasting disorder (e.g., cancer cachexia, CKD cachexia or diabetic cachexia) described herein can additional comprise administering a further compound to treat or prevent (or delay progression of) cancer, chronic kidney disease and/or diabetes. Exemplary compounds are described herein.

The present disclosure also provides a compound that inhibits VEGF-B signalling for use in the treatment of a wasting disorder (e.g., cachexia).

The present disclosure also provides a compound that inhibits VEGF-B signalling for use in the inhibition of lipolysis in a subject in need thereof.

The present disclosure also provides for use of a compound that inhibits VEGF-B signalling in the manufacture of a medicament for treating a wasting disorder (e.g., cachexia).

The present disclosure also provides for use of a compound that inhibits VEGF-B signalling in the manufacture of a medicament for inhibiting lipolysis in a subject in need thereof.

The present disclosure also provides a kit comprising a compound that inhibits VEGF-B signalling packaged with instructions for use in the treatment of a wasting disorder (e.g., cachexia).

The present disclosure further provides a kit comprising a compound that inhibits VEGF-B signalling packaged with instructions for use in inhibiting lipolysis in a subject in need thereof.

Exemplary wasting disorders and compounds are described herein and are to be taken to apply mutatis mutandis to the examples of the disclosure set out in the previous six paragraphs.

### BRIEF DESCRIPTION OF THE DRAWINGS

FIG. 1 is a series of graphical representations showing the effect of deletion of Vegfb on body weight and blood glucose levels. (A) Body weight in chow-fed mice. (B) Body weight loss during fasting. (C) Postprandial blood glucose values in chow-fed and fasted mice. Values are means±s.e.m; *P<0.05, **P<0.01, compared to wild-type (WT) mice. ###P<0.001 compared to fed wild-type mice; n=7-8 per genotype and per group.

FIG. 2 is a series of graphical representations showing the effect of deletion of Vegfb on quantification of plasma levels of (A) non-esterified fatty acids, (B) free glycerol, (C) triglycerides and (D) insulin in chow-fed and fasted mice. Values are means±s.e.m; *P<0.05, **P<0.01, compared to fed wild-type (WT) mice and #P<0.05, ###P<0.001 compared to fasted wild-type mice. ¶P<0.05 is comparison between fed WT mice and fed Vegfb^{-/-} mice; n=10-16 per genotype for fed mice and n=3-14 per genotype for fasted mice.

FIG. 3 is a graphical representation showing the effect of deletion of Vegfb on ex vivo lipolysis rate in visceral epididymal adipose tissue from chow-fed and fasted mice. Lipolysis was measured both in the absence or presence of forskolin (“stimulated fed”) or Atglistatin (“inhibited fed”) Values are means±s.e.m. **P<0.01, compared to WT. n=8 per genotype and per group

FIG. 4 is a graphical representation showing effect of deletion of Vegfb on relative mRNA expression of Atgl and Lipe in visceral epididymal adipose tissue from chow-fed and fasted mice. Values are means±s.e.m. *P<0.05, compared to fasted WT mice, and #P<0.05 compared to fed WT mice; n=3-5 per genotype and per group.

FIG. 5 is a series of graphical representation showing effect of Vegfb deletion on hepatic lipid accumulation. Quantification of (A) neutral lipids by Oil red 0 staining and (B) lipid droplets by immunolabeling of adipophilin in liver sections of chow-fed and fasted mice. (C) Quantification of Oil red 0 staining of heart sections from chow-fed and fasted mice. Values are means±s.e.m; ¶P<0.05, ¶¶P<0.01, ###P<0.001, *P<0.05, **P<0.01 and ***P<0.001; a.u, arbitrary units; n=3-5 per genotype and per group.

FIG. 6 is a series of graphical representations showing hepatic VEGF-B expression and signaling in chow-fed and fasted mice. (A) Relative mRNA expression of Vegfb in livers from chow-fed and fasted mice and Cpt1 in quadriceps from WT mice. (B) Relative mRNA expression of Nrp1 and Vegfr1 in livers from chow-fed and fasted mice. Values are means±s.e.m; \hat{ }\hat{ }\hat{ }P<0.001 compared to WT quadriceps and *P<0.05 compared to WT fed livers; n=3-5 per genotype and per group.

FIG. 7 is a graphical representation showing relative hepatic mRNA expression of fatty acid (FA) handling proteins in livers from chow-fed and fasted mice. Values are means±s.e.m; ¶P<0.05, #P<0.05, ##P<0.01 and ###P<0.001. n=3-5 per genotype and per group.

FIG. 8 is a graphical representation showing the effect of specific ablation of Vegfb in adipocytes on ex vivo lipolysis rate in visceral epididymal adipose tissue from chow-fed and fasted AdiCre/Vegfb Flox+, WT/WT, WT/Vegfb Flox+ and AdiCre/WT mice. Lipolysis was measured both in the absence or presence of forskolin (“stimulated”) or Atglistatin (“inhibited”). Values are means±s.e.m. *P<0.05, **P<0.01, ***P<0.001, compared to AdiCre/Vegfb Flox+; n=5 per genotype and per group.

FIG. 9 is a series of graphical representations showing effect of anti-VEGF-B treatment using 2H10 on body weight and blood glucose levels. (A) Bodyweight in chow-fed mice. (B) Body weight loss during fasting. (C) Postprandial blood glucose values in chow-fed and fasted mice. Values are means±s.e.m. ###P<0.001 compared to control treated chow-fed mice; n=8 per treatment and per group.

FIG. 10 is a series of graphical representations showing effect of anti-VEGF-B treatment using 2H10 on plasma levels of (A) non-esterified fatty acids, (B) free glycerol, (C) triglycerides and (D) insulin in chow-fed or fasted mice. Lipolysis was induced by subjecting animals to o/n fasting. Values are means±s.e.m; **P<0.01, compared to control treated fasted mice and #P<0.05, ##P<0.01, ###P<0.001 compared to control treated chow-fed mice. ¶P<0.05 is comparison between control treated chow-fed mice and anti-VEGF-B treated chow-fed mice; n=8/treatment and group.

FIG. 11 is a graphical representation showing effect of anti-VEGF-B treatment using 2H10 on ex vivo lipolysis rate in visceral epididymal adipose tissue from chow-fed and fasted wild-type and Vegfb^{-/-} mice. Lipolysis rate was measured both in the absence (“basal”), or presence, of forskolin (“stimulated fed”) or Atglistatin (“inhibited fed”). Values are means±s.e.m; **P<0.01, compared to control treated; n=8 per treatment and per group.

FIG. 12 is a graphical representation showing effect of anti-VEGF-B treatment using 2H10 on quantification of lipid droplets in liver sections from chow-fed or fasted mice. Values are means±s.e.m; p-values are: ¶¶P<0.01, ###P<0.001 and *P<0.05; n=8 per treatment and per group.

### KEY TO SEQUENCE LISTING

SEQ ID NO: 1 is an amino acid sequence of a human VEGF-B₁₈₆ isoform containing a 21 amino acid N-terminal signal sequence
SEQ ID NO: 2 is an amino acid sequence of a human VEGF-B₁₆₇ isoform containing a 21 amino acid N-terminal signal sequence
SEQ ID NO: 3 is an amino acid sequence from a V_{H} of antibody 2H10.
SEQ ID NO: 4 is an amino acid sequence from a V_{L} of antibody 2H10.
SEQ ID NO: 5 is an amino acid sequence from a V_{H} of a humanized form of antibody 2H10.
SEQ ID NO: 6 is an amino acid sequence of a V_{L} of a humanized form of antibody 2H10.
SEQ ID NO: 7 is an amino acid sequence from a V_{H} of antibody 4E12.
SEQ ID NO: 8 is an amino acid sequence of a V_{L} of antibody 4E12.
SEQ ID NO: 9 is an amino acid sequence from a V_{H} of antibody 2F5.
SEQ ID NO: 10 is an amino acid sequence of a V_{L} of antibody 2F5.
SEQ ID NO: 11 is a nucleotide sequence from a V_{L} CDR1 of antibody 2H10
SEQ ID NO: 12 is a nucleotide sequence from a V_{L} CDR2 of antibody 2H10
SEQ ID NO: 13 is a nucleotide sequence from a V_{L} CDR3 of antibody 2H10
SEQ ID NO: 14 is a nucleotide sequence from a V_{H} CDR1 of antibody 2H10
SEQ ID NO: 15 is a nucleotide sequence from a V_{H} CDR2 of antibody 2H10
SEQ ID NO: 16 is a nucleotide sequence from a V_{H} CDR3 of antibody 2H10
SEQ ID NO: 17 is an amino acid sequence from a V_{L} CDR1 of antibody 2H10
SEQ ID NO: 18 is an amino acid sequence from a V_{L} CDR2 of antibody 2H10
SEQ ID NO: 19 is an amino acid sequence from a V_{L} CDR3 of antibody 2H10
SEQ ID NO: 20 is an amino acid sequence from a V_{H} CDR1 of antibody 2H10
SEQ ID NO: 21 is an amino acid sequence from a V_{H} CDR2 of antibody 2H10
SEQ ID NO: 22 is an amino acid sequence from a V_{H} CDR3 of antibody 2H10
SEQ ID NO: 23 is a nucleotide sequence from a V_{L} CDR1 of antibody 2F5
SEQ ID NO: 24 is a nucleotide sequence from a V_{L} CDR2 of antibody 2F5
SEQ ID NO: 25 is a nucleotide sequence from a V_{L} CDR3 of antibody 2F5
SEQ ID NO: 26 is a nucleotide sequence from a V_{H} CDR1 of antibody 2F5
SEQ ID NO: 27 is a nucleotide sequence from a V_{H} CDR2 of antibody 2F5
SEQ ID NO: 28 is a nucleotide sequence from a V_{H} CDR3 of antibody 2F5
SEQ ID NO: 29 is an amino acid sequence from a V_{L} CDR1 of antibody 2F5
SEQ ID NO: 30 is an amino acid sequence from a V_{L} CDR2 of antibody 2F5
SEQ ID NO: 31 is an amino acid sequence from a V_{L} CDR3 of antibody 2F5
SEQ ID NO: 32 is an amino acid sequence from a V_{H} CDR1 of antibody 2F5
SEQ ID NO: 33 is an amino acid sequence from a V_{H} CDR2 of antibody 2F5
SEQ ID NO: 34 is an amino acid sequence from a V_{H} CDR3 of antibody 2F5
SEQ ID NO: 35 is a nucleotide sequence from a V_{L} CDR1 of antibody 4E12
SEQ ID NO: 36 is a nucleotide sequence from a V_{L} CDR2 of antibody 4E12
SEQ ID NO: 37 is a nucleotide sequence from a V_{L} CDR3 of antibody 4E12
SEQ ID NO: 38 is a nucleotide sequence from a V_{H} CDR1 of antibody 4E12
SEQ ID NO: 39 is a nucleotide sequence from a V_{H} CDR2 of antibody 4E12
SEQ ID NO: 40 is a nucleotide sequence from a V_{H} CDR3 of antibody 4E12
SEQ ID NO: 41 is an amino acid sequence from a V_{L} CDR1 of antibody 4E12
SEQ ID NO: 42 is an amino acid sequence from a V_{L} CDR2 of antibody 4E12
SEQ ID NO: 43 is an amino acid sequence from a V_{L} CDR3 of antibody 4E12
SEQ ID NO: 44 is an amino acid sequence from a V_{H} CDR1 of antibody 4E12
SEQ ID NO: 45 is an amino acid sequence from a V_{H} CDR2 of antibody 4E12
SEQ ID NO: 46 is an amino acid sequence from a V_{H} CDR3 of antibody 4E12

### DESCRIPTION

### General

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

Any discussion of a protein or antibody herein will be understood to include any variants of the protein or antibody produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” and/or have part or all of a signal sequence incompletely processed and, as a consequence, remain at the terminus of the antibody. It is understood that a composition comprising a particular amino acid sequence may be a heterogeneous mixture of the stated or encoded sequence and/or variants of that stated or encoded sequence.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

VEGF-B is known to exist in two major isoforms, referred to as VEGF-B₁₈₆ and VEGF-B₁₆₇. For the purposes of nomenclature only and not limitation exemplary sequences of human VEGF-B₁₈₆ is set out in NCBI Reference Sequence: NP_003368.1, in NCBI protein accession numbers NP_003368, P49765 and AAL79001 and in SEQ ID NO: 1. In the context of the present disclosure, the sequence of VEGF-B₁₈₆ can lack the 21 amino acid N-terminal signal sequence (e.g., as set out at amino acids 1 to 21 of SEQ ID NO: 1. For the purposes of nomenclature only and not limitation exemplary sequences of human VEGF-B167 is set out in NCBI Reference Sequence: NP_001230662.1, in NCBI protein accession numbers AAL79000 and AAB06274 and in SEQ ID NO: 2. In the context of the present disclosure, the sequence of VEGF-B₁₆₇ can lack the 21 amino acid N-terminal signal sequence (e.g., as set out at amino acids 1 to 21 of SEQ ID NO: 2. Additional sequence of VEGF-B can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)). Reference to human VEGF-B may be abbreviated to hVEGF-B. In one example, reference herein to VEGF-B is to VEGF-B₁₆₇ isoform.

Reference herein to VEGF-B also encompasses the VEGF-B₁₀₋₁₀₈ peptide as described in WO2006/012688.

As used herein, the term “wasting disorder” (also known as “wasting disease” or “wasting syndrome”) shall be understood to mean a disorder which involves, results at least in part from, or includes loss of weight, muscle atrophy, fatigue, weakness in someone who is not actively trying to lose weight. Wasting disorders are commonly characterized by inadvertent and/or uncontrolled (in the absence of medical intervention) loss of muscle and fat.

The term “cachexia” as used herein shall be understood to refer to a complex metabolic condition associated with an underlying (or another) condition, wherein cachexia is characterized by loss of body weight and loss of muscle with loss of fat mass. Cachexia is generally associated with increased protein catabolism due to underlying disease(s). As used herein, the term “cachexia” encompasses all stages of cachexia, including “pre-cachexia”, “overt cachexia” (also known as cachexia) and “refractory cachexia”.

The term “cancer cachexia”, also known as “cancer anorexia cachexia” shall be understood to refer to cachexia that is associated with cancer or occurring in a subject that is suffering from cancer and is characterised by an ongoing loss of muscle mass (with loss of fat mass), leading to progressive functional impairment which cannot be fully reversed by normal nutritional support.

The term “chronic kidney disease (CKD) cachexia” shall be understood to refer to cachexia that is associated with CKD or occurring in a subject that is suffering from CKD and is characterised by anorexia, increased energy expenditure, decreased protein stores characterized by a low serum albumin, and loss of body weight and loss of muscle and fat mass.

The term “diabetic cachexia” (also known as “diabetic neuropathic cachexia”) shall be understood to refer to cachexia that is associated with diabetes or occurring in a subject that is suffering from diabetes mellitus and is characterised by bilateral, painful neuropathy over the limbs and trunk, with dramatic weight loss.

The term “unintended body weight loss” refers to a condition where the subject is incapable of maintaining a healthy body weight or loses a considerable amount of body weight, without actually attempting to reduce body weight. For example a body mass index (BMI) of less than 18.5 (or any another BMI range defined by a medical specialist) is considered underweight. For the purposes of the present disclosure, the term “body mass index” or “BMI” is calculated by the following formula: mass (kg)/(height (m)²).

The term “total body mass” will be understood to mean a subject's weight.

The term “anorexia” as used herein, refers to a loss of appetite, either by medical or psychological factors, resulting in food restriction.

The term “lipolysis” shall be understood to refer to the breakdown of fats and other lipids by hydrolysis to release glycerol and free fatty acids.

The term “recombinant” shall be understood to mean the product of artificial genetic recombination. Accordingly, in the context of a recombinant protein comprising an antibody variable region, this term does not encompass an antibody naturally-occurring within a subject's body that is the product of natural recombination that occurs during B cell maturation. However, if such an antibody is isolated, it is to be considered an isolated protein comprising an antibody variable region. Similarly, if nucleic acid encoding the protein is isolated and expressed using recombinant means, the resulting protein is a recombinant protein comprising an antibody variable region. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The skilled artisan will be aware that an “antibody” is generally considered to be a protein that comprises a variable region made up of a plurality of polypeptide chains, e.g., a polypeptide comprising a light chain variable region (V_{L}) and a polypeptide comprising a heavy chain variable region (V_{H}). An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies, synhumanized antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196: 901-917, 1987; Chothia et al. Nature 342, 877-883, 1989; and/or Al-Lazikani et al., J Mol Biol 273: 927-948, 1997; the IMGT numbering system of Lefranc et al., Devel. And Compar. Immunol., 27: 55-77, 2003; or the AHO numbering system of Honnegher and Plükthun J. Mol. Biol., 309: 657-670, 2001.

“Framework regions” (FRs) are those variable domain residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an antibody, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a protein or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” or “binds specifically” shall be taken to mean that a protein of the disclosure reacts or associates more frequently, more rapidly, with greater duration and/or with greater affinity with a particular antigen or cell expressing same than it does with alternative antigens or cells. For example, a protein binds to VEGF-B with materially greater affinity (e.g., 20 fold or 40 fold or 60 fold or 80 fold to 100 fold or 150 fold or 200 fold) than it does to other growth factor (e.g., VEGF-A) or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

As used herein, the term “neutralize” shall be taken to mean that a protein is capable of blocking, reducing or preventing VEGF-B-signaling in a cell through the VEGF-R1. Methods for determining neutralization are known in the art and/or described herein.

As used herein, the term “inhibit” or “inhibiting” in the context of lipolysis shall be understood to mean that a protein described here reduces or decreases the level of lipolysis. It will be apparent from the foregoing that the protein of the present disclosure need not completely inhibit lipolysis, rather it need only reduce lipolysis by a statistically significant amount, for example, by at least about 10%, or about 20%, or about 30%, or about 40%, or about 50%, or about 60%, or about 70%, or about 80%, or about 90%, or about 95%. Methods for determining inhibition of lipolysis are known in the art and/or described herein.

As used herein, the terms “treating”, “treat” or “treatment” include administering a protein described herein to thereby reduce or eliminate at least one symptom of a specified disease or condition or to slow progression of the disease or condition.

As used herein, the term “subject” shall be taken to mean any animal including humans, for example a mammal. Exemplary subjects include but are not limited to humans and non-human primates. For example, the subject is a human.

### Treatment of a Wasting Disorder

The present disclosure provides, for example, a method of treating a wasting disorder in a subject comprising administering to the subject a compound that inhibits VEGF-B signaling.

In one example, the subject suffers from a wasting disorder. For example, the wasting disorder is selected from the group consisting of cachexia, unintended body weight loss, fat wasting and anorexia.

In one example, the wasting disorder is cachexia. For example, the cachexia can be pre-cachexia, overt cachexia (or cachexia) or refractory cachexia. In one example, the subject suffers from cachexia. The different stages of cachexia can be diagnosed based on the following clinically acceptable criteria:

### Pre-Cachexia

Weight loss ≤5%; and/or
Anorexia and metabolic changes.

### Cachexia or Overt Cachexia

Weight loss >5% over a period of six months (in the absence of starvation);
A BMI <20 together with weight loss;
Weight loss >2% together with an appendicular skeletal muscle index consistent with sarcopenia (males <7.26 kg/m2; females <5.45 kg/m2); and/or
Reduced food intake with or without systemic inflammation.

### Refractory Cachexia

Weight loss non-responsive to treatment;
<3 months expected survival.

In one example, the cachexia is cancer cachexia. For example, the subject is suffering from cancer cachexia. For example, the cancer is a solid tumor, such as a sarcoma or carcinoma. For example, the carcinoma includes, but is not limited to, a carcinoma of the prostate, ovary, breast, lung, liver, kidney, colon, pancreas, gastrointestinal tract or stomach. In one example, the cancer is a non-solid tumor, for example leukemia or lymphoma. In one example, the subject suffers from a stage 0 cancer. For example, the carcinoma is in situ. In another example, the subject suffers from a stage I, II or III cancer. For example, the carcinoma has spread beyond the organ of origin to nearby lymph nodes and/or tissues or organs adjacent to the location of the primary tumor. In one example, the subject suffers from a stage IV cancer. For example, the cancer has spread to distant tissues and/or organs.

In one example, the cachexia is chronic kidney disease cachexia. For example, the subject is suffering from chronic kidney disease cachexia.

In one example, the cachexia is diabetic cachexia. For example, the subject is suffering from diabetic cachexia. For example, a subject suffering from diabetic cachexia has a clinically accepted marker of diabetes, such as:
Fasting plasma glucose of greater than or equal to 7 nmol/L or 126 mg/dl;
Casual plasma glucose (taken at any time of the day) of greater than or equal to 11.1 nmol/L or 200 mg/dl with the symptoms of diabetes.
Oral glucose tolerance test (OGTT) value of greater than or equal to 11.1 nmol/L or 200 mg/dl measured at a two-hour interval. The OGTT is given over a two or three-hour time span.

In one example, the subject suffers from type 1 diabetes. For example, the subject suffers from cachexia associated with type 1 diabetes.

In one example, the subject suffers from type 2 diabetes. For example, the subject suffers from cachexia associated with type 2 diabetes.

In one example, performing a method described herein according to any example of the disclosure results in enhancement of a clinical response and/or delayed disease progression.

By “clinical response” is meant an improvement in the symptoms of disease. The clinical response may be achieved within a certain time frame, for example, within or at about 8 weeks from the start of treatment with, or from the initial administration. Clinical response may also be sustained for a period of time, such as for >24 weeks, or ≥48 weeks.

Methods of the present disclosure achieve one or more of the following effects:
Reduce or prevent lipolysis;
Reduce or prevent hepatic lipid accumulation;
Reduce or prevent an increase in plasma non-esterified fatty acid levels; and/or
Reduce or prevent an increase in plasma free glycerol levels.

Methods for quantitative assessment of the above parameters are known in the art and/or described herein.

As will be apparent to the skilled person a “reduction” in an effect in a subject will be comparative to another subject who also suffers from a wasting disorder but who has not received treatment with a method described herein. This does not necessarily require a side-by-side comparison of two subjects. Rather population data can be relied upon. For example a population of subjects suffering from a wasting disorder who have not received treatment with a method described herein (optionally, a population of similar subjects to the treated subject, e.g., age, weight) are assessed and the mean values are compared to results of a subject or population of subjects treated with a method described herein.

### VEGF-B Signaling Inhibitors

### Proteins Comprising Antibody Variable Regions

An exemplary VEGF-B signaling inhibitor comprises an antibody variable region, e.g., is an antibody or an antibody fragment that binds to VEGF-B and neutralizes VEGF-B signaling.

In one example, the antibody variable region binds specifically to VEGF-B.

Suitable antibodies and proteins comprising variable regions thereof are known in the art.

For example, anti-VEGF-B antibodies and fragments thereof are described in WO2006/012688.

In one example, the anti-VEGF-B antibody or fragment thereof is an antibody that competitively inhibits the binding of 2H10 to VEGF-B or an antigen binding fragment thereof. In one example, the anti-VEGF-B antibody or fragment thereof is antibody 2H10 or a chimeric, CDR grafted or humanized version thereof or an antigen binding fragment thereof. In this regard, antibody 2H10 comprises a V_{H} comprising a sequence set forth in SEQ ID NO: 3 and a V_{L} comprising a sequence set forth in SEQ ID NO: 4. Exemplary chimeric and humanized versions of this antibody are described in WO2006/012688.

In one example, the anti-VEGF-B antibody or fragment thereof comprises a V_{H} comprising a sequence set forth in SEQ ID NO: 5 and a V_{L} comprising a sequence set forth in SEQ ID NO: 6.

In one example, the anti-VEGF-B antibody or fragment thereof is an antibody that competitively inhibits the binding of 4E12 to VEGF-B or an antigen binding fragment thereof. In one example, the anti-VEGF-B antibody or fragment thereof is antibody 4E12 or a chimeric, CDR grafted or humanized version thereof or an antigen binding fragment thereof. In this regard, antibody 4E12 comprises a V_{H} comprising a sequence set forth in SEQ ID NO: 7 and a V_{L} comprising a sequence set forth in SEQ ID NO: 8.

In one example, the compound is a protein comprising a humanized variable region of antibody 4E12. For example, the protein comprises a variable region comprising the complementarity determining regions (CDRs) of the V_{H} and/or the V_{L} of antibody 4E12. For example, the protein comprises:

(i) a V_{H} comprising:
(a) a CDR1 comprising a sequence set forth in amino acids 25-34 of SEQ ID NO: 7;
(b) a CDR2 comprising a sequence set forth in amino acids 49-65 of SEQ ID NO: 7; and
(c) a CDR3 comprising a sequence set forth in amino acids 98-105 of SEQ ID NO: 7; and/or
   (ii) a V_{L} comprising:
(a) a CDR1 comprising a sequence set forth in amino acids 24-34 of SEQ ID NO: 8;
(b) a CDR2 comprising a sequence set forth in amino acids 50-56 of SEQ ID NO: 8; and
(c) a CDR3 comprising a sequence set forth in amino acids 89-97 of SEQ ID NO: 8.

In one example, the anti-VEGF-B antibody or fragment thereof is an antibody that competitively inhibits the binding of 2F5 to VEGF-B or an antigen binding fragment thereof. In one example, the anti-VEGF-B antibody or fragment thereof is antibody 2F5 or a chimeric, CDR grafted or humanized version thereof or an antigen binding fragment thereof. In this regard, antibody 2E5 comprises a V_{H} comprising a sequence set forth in SEQ ID NO: 9 and a V_{L} comprising a sequence set forth in SEQ ID NO: 10.

In one example, the compound is a protein comprising a humanized variable region of antibody 2F5. For example, the protein comprises a variable region comprising the complementarity determining regions (CDRs) of the V_{H} and/or the V_{L} of antibody 2F5. For example, the protein comprises:

(i) a V_{H} comprising:
(a) a CDR1 comprising a sequence set forth in amino acids 25-34 of SEQ ID NO: 9;
(b) a CDR2 comprising a sequence set forth in amino acids 49-65 of SEQ ID NO: 9; and
(c) a CDR3 comprising a sequence set forth in amino acids 98-107 of SEQ ID NO: 9; and/or
   (ii) a V_{L} comprising:
(a) a CDR1 comprising a sequence set forth in amino acids 24-34 of SEQ ID NO: 10;
(b) a CDR2 comprising a sequence set forth in amino acids 50-56 of SEQ ID NO: 10; and
(c) a CDR3 comprising a sequence set forth in amino acids 89-96 of SEQ ID NO: 10.

In another example, an antibody or protein comprising a variable region thereof is produced using a standard method, e.g., as is known in the art or briefly described herein.

### Immunization-Based Methods

To generate antibodies, VEGF-B or an epitope bearing fragment or portion thereof or a modified form thereof or nucleic acid encoding same (an “immunogen”), optionally formulated with any suitable or desired adjuvant and/or pharmaceutically acceptable carrier, is administered to a subject (for example, a non-human animal subject, such as, a mouse, a rat, a chicken etc.) in the form of an injectable composition. Exemplary non-human animals are mammals, such as murine animals (e.g., rats or mice). Injection may be intranasal, intramuscular, sub-cutaneous, intravenous, intradermal, intraperitoneal, or by other known route. Optionally, the immunogen is administered numerous times. Means for preparing and characterizing antibodies are known in the art (See, e.g., Antibodies: A Laboratory Manual, Cold Spring Harbor Laboratory, 1988). Methods for producing anti-VEGF-B antibodies in mice are described in WO2006/012688.

The production of polyclonal antibodies may be monitored by sampling blood of the immunized animal at various points following immunization. A second, booster injection, may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are exemplary antibodies contemplated by the present disclosure. Generally, production of monoclonal antibodies involves, immunizing a subject (e.g., a rodent, e.g., mouse or rat) with the immunogen under conditions sufficient to stimulate antibody producing cells. In some examples, a mouse genetically-engineered to express human antibodies and not express murine antibodies proteins, is immunized to produce an antibody (e.g., as described in PCT/US2007/008231 and/or Lonberg et al., Nature 368 (1994): 856-859). Following immunization, antibody producing somatic cells (e.g., B lymphocytes) are fused with immortal cells, e.g., immortal myeloma cells. Various methods for producing such fused cells (hybridomas) are known in the art and described, for example, in Kohler and Milstein, Nature 256, 495-497, 1975. The hybridoma cells can then be cultured under conditions sufficient for antibody production.

The present disclosure contemplates other methods for producing antibodies, e.g., ABL-MYC technology (as described, for example in Largaespada et al, Curr. Top. Microbiol. Immunol, 166, 91-96. 1990).

### Library-Based Methods

The present disclosure also encompasses screening of libraries of antibodies or proteins comprising antigen binding domains thereof (e.g., comprising variable regions thereof) to identify a VEGF-B binding antibody or protein comprising a variable region thereof.

Examples of libraries contemplated by this disclosure include naïve libraries (from unchallenged subjects), immunized libraries (from subjects immunized with an antigen) or synthetic libraries. Nucleic acid encoding antibodies or regions thereof (e.g., variable regions) are cloned by conventional techniques (e.g., as disclosed in Sambrook and Russell, eds, Molecular Cloning: A Laboratory Manual, 3rd Ed, vols. 1-3, Cold Spring Harbor Laboratory Press, 2001) and used to encode and display proteins using a method known in the art. Other techniques for producing libraries of proteins are described in, for example in U.S. Pat. No. 6,300,064 (e.g., a HuCAL library of Morphosys AG); U.S. Pat. Nos. 5,885,793; 6,204,023; 6,291,158; or U.S. Pat. No. 6,248,516.

The proteins according to the disclosure may be soluble secreted proteins or may be presented as a fusion protein on the surface of a cell, or particle (e.g., a phage or other virus, a ribosome or a spore). Various display library formats are known in the art. For example, the library is an in vitro display library (e.g., a ribosome display library, a covalent display library or a mRNA display library, e.g., as described in U.S. Pat. No. 7,270,969). In yet another example, the display library is a phage display library wherein proteins comprising antigen binding domains of antibodies are expressed on phage, e.g., as described in U.S. Pat. Nos. 6,300,064; 5,885,793; 6,204,023; 6,291,158; or U.S. Pat. No. 6,248,516. Other phage display methods are known in the art and are contemplated by the present disclosure. Similarly, methods of cell display are contemplated by the disclosure, e.g., bacterial display libraries, e.g., as described in U.S. Pat. No. 5,516,637; yeast display libraries, e.g., as described in U.S. Pat. No. 6,423,538 or a mammalian display library.

Methods for screening display libraries are known in the art. In one example, a display library of the present disclosure is screened using affinity purification, e.g., as described in Scopes (In: Protein purification: principles and practice, Third Edition, Springer Verlag, 1994). Methods of affinity purification typically involve contacting proteins comprising antigen binding domains displayed by the library with a target antigen (e.g., VEGF-B) and, following washing, eluting those domains that remain bound to the antigen.

Any variable regions or scFvs identified by screening are readily modified into a complete antibody, if desired. Exemplary methods for modifying or reformatting variable regions or scFvs into a complete antibody are described, for example, in Jones et al., J Immunol Methods. 354:85-90, 2010; or Jostock et al., J Immunol Methods, 289: 65-80, 2004. Alternatively, or additionally, standard cloning methods are used, e.g., as described in Ausubel et al (In: Current Protocols in Molecular Biology. Wiley Interscience, ISBN 047 150338, 1987), and/or (Sambrook et al (In: Molecular Cloning: Molecular Cloning: A Laboratory Manual, Cold Spring Harbor Laboratories, New York, Third Edition 2001).

### Deimmunized, Chimeric, Humanized, Synhumanized, Primatized and Human Proteins

The proteins of the present disclosure may be a humanized protein.

The term “humanized protein” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized proteins also include proteins in which one or more residues of the human protein are modified by one or more amino acid substitutions and/or one or more FR residues of the human protein are replaced by corresponding non-human residues. Humanized proteins may also comprise residues which are found in neither the human antibody or in the non-human antibody. Any additional regions of the protein (e.g., Fc region) are generally human. Humanization can be performed using a method known in the art, e.g., U.S. Pat. Nos. 5,225,539, 6,054,297, 7,566,771 or U.S. Pat. No. 5,585,089. The term “humanized protein” also encompasses a super-humanized protein, e.g., as described in U.S. Pat. No. 7,732,578.

The proteins of the present disclosure may be human proteins. The term “human protein” as used herein refers to proteins having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human protein will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516.

The proteins of the present disclosure may be synhumanized proteins. The term “synhumanized protein” refers to a protein prepared by a method described in WO2007/019620. A synhumanized protein includes a variable region of an antibody, wherein the variable region comprises FRs from a New World primate antibody variable region and CDRs from a non-New World primate antibody variable region. For example, a synhumanized protein includes a variable region of an antibody, wherein the variable region comprises FRs from a New World primate antibody variable region and CDRs from a mouse or rat antibody.

The proteins of the present disclosure may be primatized proteins. A “primatized protein” comprises variable region(s) from an antibody generated following immunization of a non-human primate (e.g., a cynomolgus macaque). Optionally, the variable regions of the non-human primate antibody are linked to human constant regions to produce a primatized antibody. Exemplary methods for producing primatized antibodies are described in U.S. Pat. No. 6,113,898.

In one example a protein of the disclosure is a chimeric protein. The term “chimeric proteins” refers to proteins in which an antigen binding domain is from a particular species (e.g., murine, such as mouse or rat) or belonging to a particular antibody class or subclass, while the remainder of the protein is from a protein derived from another species (such as, for example, human or non-human primate) or belonging to another antibody class or subclass. In one example, a chimeric protein is a chimeric antibody comprising a V_{H} and/or a V_{L} from a non-human antibody (e.g., a murine antibody) and the remaining regions of the antibody are from a human antibody. The production of such chimeric proteins is known in the art, and may be achieved by standard means (as described, e.g., in U.S. Pat. Nos. 6,331,415; 5,807,715; 4,816,567 and 4,816,397).

The present disclosure also contemplates a deimmunized protein, e.g., as described in WO2000/34317 and WO2004/108158. De-immunized antibodies and proteins have one or more epitopes, e.g., B cell epitopes or T cell epitopes removed (i.e., mutated) to thereby reduce the likelihood that a subject will raise an immune response against the antibody or protein.

### Other Proteins Comprising Antibody Variable Regions

The present disclosure also contemplates other proteins comprising a variable region or antigen binding domain of an antibody, such as:

(i) a single-domain antibody, which is a single polypeptide chain comprising all or a portion of the V_{H} or a V_{L} of an antibody (see, e.g., U.S. Pat. No. 6,248,516);
(ii) diabodies, triabodies and tetrabodies, e.g., as described in U.S. Pat. No. 5,844,094 and/or US2008152586;
(iii) scFvs, e.g., as described in U.S. Pat. No. 5,260,203;
(iv) minibodies, e.g., as described in U.S. Pat. No. 5,837,821;
(v) “key and hole” bispecific proteins as described in U.S. Pat. No. 5,731,168;
(vi) heteroconjugate proteins, e.g., as described in U.S. Pat. No. 4,676,980;
(vii) heteroconjugate proteins produced using a chemical cross-linker, e.g., as described in U.S. Pat. No. 4,676,980;
(viii) Fab′-SH fragments, e.g., as described in Shalaby et al, J. Exp. Med., 175: 217-225, 1992; or
(ix) Fab₃ (e.g., as described in EP19930302894).

### Constant Domain Fusions

The present disclosure encompasses a protein comprising a variable region of an antibody and a constant region or Fc or a domain thereof, e.g., C_{H}2 and/or C_{H}3 domain. Suitable constant regions and/or domains will be apparent to the skilled artisan and/or the sequences of such polypeptides are readily available from publicly available databases. Kabat et al also provide description of some suitable constant regions/domains.

Constant regions and/or domains thereof are useful for providing biological activities such as, dimerization, extended serum half-life e.g., by binding to FcRn (neonatal Fc Receptor), antigen dependent cell cytotoxicity (ADCC), complement dependent cytotoxicity (CDC, antigen dependent cell phagocytosis (ADCP).

The present disclosure also contemplates proteins comprising mutant constant regions or domains, e.g., as described in U.S. Pat. Nos. 7,217,797; 7,217,798; or US20090041770 (having increased half-life) or US2005037000 (increased ADCC).

### Stabilized Proteins

Neutralizing proteins of the present disclosure can comprise an IgG4 constant region or a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

### Additional Protein-Based VEGF-B Signaling Inhibitors

Other proteins that may interfere with the productive interaction of VEGF-B with its receptor include mutant VEGF-B proteins.

In one example, the inhibitor is a soluble protein comprising one or more domains of a VEGF-R1 that bind to VEGF-B (and, e.g., do not substantially bind to VEGF-A). In one example, the soluble protein additionally comprises a constant region of an antibody, such as an IgG1 antibody. For example, the soluble protein additionally comprises a Fc region and, optionally a hinge region of an antibody, e.g., an IgG1 antibody.

In one example, the protein inhibitor is an antibody mimetic, e.g., a protein scaffold comprising variable regions that bind to a target protein in a manner analogous to an antibody. A description of exemplary antibody mimetics follows.

### Immunoglobulins and Immunoglobulin Fragments

An example of a compound of the present disclosure is a protein comprising a variable region of an immunoglobulin, such as a T cell receptor or a heavy chain immunoglobulin (e.g., an IgNAR, a camelid antibody).

Heavy Chain Immunoglobulins

Heavy chain immunoglobulins differ structurally from many other forms of immunoglobulin (e.g., antibodies) in so far as they comprise a heavy chain, but do not comprise a light chain. Accordingly, these immunoglobulins are also referred to as “heavy chain only antibodies”. Heavy chain immunoglobulins are found in, for example, camelids and cartilaginous fish (also called IgNAR).

The variable regions present in naturally occurring heavy chain immunoglobulins are generally referred to as “V_{H} domains” in camelid Ig and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

Heavy chain immunoglobulins do not require the presence of light chains to bind with high affinity and with high specificity to a relevant antigen. This means that single domain binding fragments can be derived from heavy chain immunoglobulins, which are easy to express and are generally stable and soluble.

A general description of heavy chain immunoglobulins from camelids and the variable regions thereof and methods for their production and/or isolation and/or use is found inter alia in the following references WO94/04678, WO97/49805 and WO 97/49805.

A general description of heavy chain immunoglobulins from cartilaginous fish and the variable regions thereof and methods for their production and/or isolation and/or use is found inter alia in WO2005/118629.

V-Like Proteins

An example of a compound of the disclosure is a T-cell receptor. T cell receptors have two V-domains that combine into a structure similar to the Fv module of an antibody. Novotny et al., Proc Natl Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999/045110 or WO2011/107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999/045110.

Adnectins

In one example, a compound of the disclosure is an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005/056764.

Anticalins

In a further example, a compound of the disclosure is an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297B1 or US20070224633.

Affibodies

In a further example, a compound of the disclosure is an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

Avimers

In a further example, a compound of the disclosure is an Avimer. Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see WO2002088171.

DARPins

In a further example, a compound of the disclosure is a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### Methods for Producing Proteins

### Recombinant Expression

In the case of a recombinant protein, nucleic acid encoding same can be cloned into expression vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce an antibody. Exemplary cells used for expressing a protein of the disclosure are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art. See U.S. Pat. No. 4,816,567 or 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding an antibody (e.g., derived from the information provided herein), an enhancer element, a promoter, and a transcription termination sequence. The skilled artisan will be aware of suitable sequences for expression of an antibody. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA-coated tungsten or gold particles (Agracetus Inc., WI, USA) amongst others.

The host cells used to produce the antibody may be cultured in a variety of media, depending on the cell type used. Commercially available media such as Ham's Fl0 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

### Protein Purification

Following production/expression, a protein of the disclosure is purified using a method known in the art. Such purification provides the protein of the disclosure substantially free of nonspecific protein, acids, lipids, carbohydrates, and the like. In one example, the protein will be in a preparation wherein more than about 90% (e.g. 95%, 98% or 99%) of the protein in the preparation is a protein of the disclosure.

Standard methods of peptide purification are employed to obtain an isolated protein of the disclosure, including but not limited to various high-pressure (or performance) liquid chromatography (HPLC) and non-HPLC polypeptide isolation protocols, such as size exclusion chromatography, ion exchange chromatography, hydrophobic interaction chromatography, mixed mode chromatography, phase separation methods, electrophoretic separations, precipitation methods, salting in/out methods, immunochromatography, and/or other methods.

In one example, affinity purification is useful for isolating a fusion protein comprising a label. Methods for isolating a protein using affinity chromatography are known in the art and described, for example, in Scopes (In: Protein purification: principles and practice, Third Edition, Springer Verlag, 1994). For example, an antibody or compound that binds to the label (in the case of a polyhistidine tag this may be, for example, nickel-NTA) is immobilized on a solid support. A sample comprising a protein is then contacted to the immobilized antibody or compound for a time and under conditions sufficient for binding to occur. Following washing to remove any unbound or non-specifically bound protein, the protein is eluted.

In the case of a protein comprising a Fc region of an antibody, protein A or protein G or modified forms thereof can be used for affinity purification. Protein A is useful for isolating purified proteins comprising a human γ1, γ2, or γ4 heavy chain Fc region. Protein G is recommended for all mouse Fc isotypes and for human γ3.

### Nucleic Acid-Based VEGF-B Signaling Inhibitors

In one example of the disclosure, therapeutic methods as described herein according to any example of the disclosure involve reducing expression of VEGF-B. For example, such a method involves administering a compound that reduces transcription and/or translation of the nucleic acid. In one example, the compound is a nucleic acid, e.g., an antisense polynucleotide, a ribozyme, a PNA, an interfering RNA, a siRNA, a microRNA

### Antisense Nucleic Acids

The term “antisense nucleic acid” shall be taken to mean a DNA or RNA or derivative thereof (e.g., LNA or PNA), or combination thereof that is complementary to at least a portion of a specific mRNA molecule encoding a polypeptide as described herein in any example of the disclosure and capable of interfering with a post-transcriptional event such as mRNA translation. The use of antisense methods is known in the art (see for example, Hartmann and Endres (editors), Manual of Antisense Methodology, Kluwer (1999)).

An antisense nucleic acid of the disclosure will hybridize to a target nucleic acid under physiological conditions. Antisense nucleic acids include sequences that correspond to structural genes or coding regions or to sequences that effect control over gene expression or splicing. For example, the antisense nucleic acid may correspond to the targeted coding region of a nucleic acid encoding VEGF-B, or the 5′-untranslated region (UTR) or the 3′-UTR or combination of these. It may be complementary in part to intron sequences, which may be spliced out during or after transcription, for example only to exon sequences of the target gene. The length of the antisense sequence should be at least 19 contiguous nucleotides, for example, at least 50 nucleotides, such as at least 100, 200, 500 or 1000 nucleotides of a nucleic acid encoding VEGF-B. The full-length sequence complementary to the entire gene transcript may be used. The length can be 100-2000 nucleotides. The degree of identity of the antisense sequence to the targeted transcript should be at least 90%, for example, 95-100%.

Exemplary antisense nucleic acids against VEGF-B are described, for example, in WO2003/105754.

### Catalytic Nucleic Acid

The term “catalytic nucleic acid” refers to a DNA molecule or DNA-containing molecule (also known in the art as a “deoxyribozyme” or “DNAzyme”) or a RNA or RNA-containing molecule (also known as a “ribozyme” or “RNAzyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are useful in this disclosure are a hammerhead ribozyme and a hairpin ribozyme.

### RNA Interference

RNA interference (RNAi) is useful for specifically inhibiting the production of a particular protein. Without being limited by theory, this technology relies on the presence of dsRNA molecules that contain a sequence that is essentially identical to the mRNA of the gene of interest or part thereof, in this case an mRNA encoding a VEGF-B. Conveniently, the dsRNA can be produced from a single promoter in a recombinant vector host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present disclosure is well within the capacity of a person skilled in the art, particularly considering WO99/32619, WO99/53050, WO99/49029, and WO01/34815.

The length of the sense and antisense sequences that hybridize should each be at least 19 contiguous nucleotides, such as at least 30 or 50 nucleotides, for example at least 100, 200, 500 or 1000 nucleotides. The full-length sequence corresponding to the entire gene transcript may be used. The lengths can be 100-2000 nucleotides. The degree of identity of the sense and antisense sequences to the targeted transcript should be at least 85%, for example, at least 90% such as, 95-100%.

Exemplary small interfering RNA (“siRNA”) molecules comprise a nucleotide sequence that is identical to about 19-21 contiguous nucleotides of the target mRNA. For example, the siRNA sequence commences with the dinucleotide AA, comprises a GC-content of about 30-70% (for example, 30-60%, such as 40-60% for example about 45%-55%), and does not have a high percentage identity to any nucleotide sequence other than the target in the genome of the mammal in which it is to be introduced, for example as determined by standard BLAST search. Exemplary siRNA that reduce expression of VEGF-B are commercially available from Santa Cruz Biotechnology or Novus Biologicals.

Short hairpin RNA (shRNA) that reduce expression of VEGF-B are also known in the art and commercially available from Santa Cruz Biotechnology.

### Screening Assays

Compounds that inhibit VEGF-B signaling can be identified using techniques known in the art, e.g., as described below. Similarly, amounts of VEGF-B signaling inhibitors suitable for use in a method described herein can be determined or estimated using techniques known in the art, e.g., as described below.

### Neutralization Assays

For compounds that bind to VEGF-B and inhibit signaling, a neutralization assay can be used.

In one example, a neutralization assay involves contacting VEGF-B with a compound in the presence or absence of detectably labeled soluble VEGF-R1 or contacting detectably labeled VEGF-B with a compound in the presence or absence of a cell expressing VEGF-R1 or a soluble VEGF-R1. The level of VEGF-B bound to the VEGF-R1 is then assessed. A reduced level of bound VEGF-B in the presence of the compound compared to in the absence of the compound indicates the compound inhibits VEGF-B binding to VEGF-R1 and, as a consequence VEGF-B signaling.

Another neutralization assay is described in WO2006/012688 and involves contacting a fragment of VEGF-R1 comprising the second Ig-like domain immobilized on a solid support with a subsaturating concentration of recombinant VEGF-B pre-incubated with a compound. Following washing to remove unbound protein, the immobilized protein is contacted with anti-VEGF-B antibody and the amount of bound antibody (indicative of immobilized VEGF-B) determined. A compound that reduces the level of bound antibody compared to the level in the absence of the compound is considered an inhibitor of VEGF-B signaling.

In another example, a compound that inhibits VEGF-B signaling is identified using a cell dependent on VEGF-B signaling for proliferation, e.g., a BaF3 cell modified as described in WO2006/012688 to express a chimeric receptor incorporating the intracellular domain of the human erythropoietin receptor and the extracellular domain of VEGF-R1. Cells are cultured in the presence of VEGF-B and in the presence or absence of a compound. Cell proliferation is then assessed using standard methods, e.g., colony formation assays, thymidine incorporation or uptake of another suitable marker of cell proliferation (e.g., a MTS dye reduction assay). A compound that reduces the level of proliferation in the presence of VEGF-B is considered an inhibitor of VEGF-B signaling.

Compounds can also be assessed for their ability to bind to VEGF-B using standard methods. Methods for assessing binding to a protein are known in the art, e.g., as described in Scopes (In: Protein purification: principles and practice, Third Edition, Springer Verlag, 1994). Such a method generally involves labeling the compound and contacting it with immobilized VEGF-B. Following washing to remove non-specific bound compound, the amount of label and, as a consequence, bound compound is detected. Of course, the compound can be immobilized and the VEGF-B labeled. Panning-type assays can also be used. Alternatively, or additionally, surface plasmon resonance assays can be used.

### Expression Assays

A compound that reduces or prevents expression of VEGF-B is identified by contacting a cell with the compound and determining the level of expression of the VEGF-B. Suitable methods for determining gene expression at the nucleic acid level are known in the art and include, for example, quantitative polymerase chain reaction (qPCR) or microarray assays. Suitable methods for determining expression at the protein level are also known in the art and include, for example, enzyme-linked immunosorbent assay (ELISA), fluorescence linked immunosorbent assay (FLISA), immunofluorescence or Western blotting.

### In Vivo Assays

Compounds described herein can be tested for activity in animal models. In one example, the animal model is a model of fasting-induced lipolysis. For example, C57/BL6 mice are subjected to overnight fasting (e.g., 14 hours) and assessed over time in the presence or absence of a test compound (i.e., a compound that inhibits VEGF-B signalling). Parameters associated with lipolysis, including for example hepatic lipid accumulation, expression of hepatic fatty acid transporters, plasma NEFAs and glycerol and/or basal lipolysis rate are assessed and compared to animals not subjected to overnight fasting (i.e., chow-fed animals).

### Pharmaceutical Compositions and Methods of Treatment

A compound that inhibits VEGF-B signaling (syn. active ingredient) is useful for parenteral, topical, oral, or local administration, aerosol administration, or transdermal administration, or for therapeutic treatment. In one example, the compound is administered parenterally, such as subcutaneously or intravenously.

Formulation of a compound to be administered will vary according to the route of administration and formulation (e.g., solution, emulsion, capsule) selected. An appropriate pharmaceutical composition comprising compound to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The compound can be lyophilized for storage and reconstituted in a suitable carrier prior to use according to art-known lyophilization and reconstitution techniques.

The optimum concentration of the active ingredient(s) in the chosen medium can be determined empirically, according to procedures known to the skilled artisan, and will depend on the ultimate pharmaceutical formulation desired.

The dosage ranges for the administration of the compound of the disclosure are those large enough to produce the desired effect. For example, the composition comprises a therapeutically effective amount of the compound.

As used herein, the term “effective amount” shall be taken to mean a sufficient quantity of the compound to inhibit/reduce/prevent signaling of VEGF-B in a subject. The skilled artisan will be aware that such an amount will vary depending on, for example, the compound and/or the particular subject and/or the type and/or the severity of cachexia being treated. Accordingly, this term is not to be construed to limit the disclosure to a specific quantity, e.g., weight or number of compounds.

As used herein, the term “therapeutically effective amount” shall be taken to mean a sufficient quantity of compound to reduce or inhibit one or more symptoms of a wasting disorder.

In one example, the compound is administered in an amount effective to have one or more of the following effects:
Reduce or prevent lipolysis;
Reduce or prevent hepatic lipid accumulation;
Reduce or prevent an increase in plasma non-esterified fatty acid levels; and/or
Reduce or prevent an increase in plasma free glycerol levels.

The dosage should not be so large as to cause adverse side effects, such as hyper viscosity syndromes, pulmonary edema, congestive heart failure, and the like.

Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the compound is administered at an initial (or loading) dose which is higher than subsequent (maintenance doses). For example, the compound is administered at an initial dose of between about 1 mg/kg to about 30 mg/kg. The compound is then administered at a maintenance dose of between about 0.0001 mg/kg to about 1 mg/kg. The maintenance doses may be administered every 7-35 days, such as, every 14 or 21 or 28 days.

In some examples, a dose escalation regime is used, in which a compound is initially administered at a lower dose than used in subsequent doses. This dosage regime is useful in the case of subject's initially suffering adverse events

In the case of a subject that is not adequately responding to treatment, multiple doses in a week may be administered. Alternatively, or in addition, increasing doses may be administered.

A subject may be retreated with the compound, by being given more than one exposure or set of doses, such as at least about two exposures of the compound, for example, from about 2 to 60 exposures, and more particularly about 2 to 40 exposures, most particularly, about 2 to 20 exposures.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

A method of the present disclosure may also include co-administration of a compound of the disclosure together with another therapeutically effective agent for the treatment of a wasting disorder, cancer, chronic kidney disease and/or diabetes.

In one example, the compound(s) of the disclosure is used in combination with at least one additional known compound which is currently being used or is in development for preventing or treating a wasting disorder. Exemplary compounds include orexigenic agents (i.e., appetite stimulants, such as L-carnitine, megestrol acetate, and melatonin), anabolic steroids (e.g., selective androgen receptor modulators (SARMs) such as enobosarm, espindolol and testosterone) and/or anti-inflammatory drugs (e.g., thalidomide, pentoxyphylline, a monoclonal antibody against interleukin-1α, ghrelin and the ghrelin agonist anamorelin).

In one example, the compound(s) of the disclosure is used in combination with at least one additional known compound which is currently being used or is in development for preventing or treating a cancer.

In one example, the additional therapeutic agent for preventing or treating a cancer is a chemotherapeutic agent. Exemplary chemotherapy agents include, for example, caboplatin, cytarabine, chlorambucil, cisplatin, cyclophosphamide, danorubicin, docetaxal, doxorubicin, erlotinib, etoposide, fluorouracil, fludarabine, idarubicin, irinotecan, methotrexate, mitoxantrone, paclitaxel, topotecan, vincristine and vinblastine.

In one example, the additional therapeutic agent for preventing or treating a cancer is a therapeutic antibody. Exemplary therapeutic antibodies are known to the skilled person and include, but are not limited to, Abagovomab; Abciximab; Abituzumab; Abrilumab; Actoxumab; Adalimumab; Adecatumumab; Aducanumab; Afelimomab; Afutuzumab; Alacizumab pegol; Alemtuzumab; Alirocumab; Altumomab pentetate; Amatuximab; Anatumomab mafenatox; Anetumab ravtansine; Anifrolumab; Anrukinzumab; Apolizumab; Arcitumomab; Ascrinvacumab; Aselizumab; Atezolizumab; Atinumab; Atlizumab (tocilizumab); Atorolimumab; Bapineuzumab; Basiliximab; Bavituximab; Bectumomab; Begelomab; Belimumab; Benralizumab; Bertilimumab; Besilesomab; Bevacizumab; Bezlotoxumab; Biciromab; Bimagrumab; Bimekizumab; Bivatuzumab mertansine; Blinatumomab; Blosozumab; Bococizumab; Brentuxim abvedotin; Briakinumab; Brodalumab; Brolucizumab; Brontictuzumab; Canakinumab; Cantuzumab mertansine; Cantuzumab ravtansine; Caplacizumab; Capromab pendetide; Carlumab; Catumaxomab; cBR96-doxorubicin immunoconjugate; Cedelizumab; Certolizumab pegol; Cetuximab; Citatuzumab bogatox; Cixutumumab; Clazakizumab; Clenoliximab; Clivatuzumab tetraxetan; Codrituzumab; Coltuximab ravtansine; Conatumumab; Concizumab; Crenezumab; Dacetuzumab; Daclizumab; Dalotuzumab; Dapirolizumab pegol; Daratumumab; Dectrekumab; Demcizumab; Denintuzumab mafodotin; Denosumab; Derlotuximab biotin; Detumomab; Dinutuximab; Diridavumab; Dorlimomab aritox; Drozitumab; Duligotumab; Dupilumab; Durvalumab; Dusigitumab; Ecromeximab; Eculizumab; Edobacomab; Edrecolomab; Efalizumab; Efungumab; Eldelumab; Elgemtumab; Elotuzumab; Elsilimomab; Emactuzumab; Emibetuzumab; Enavatuzumab; Enfortumab vedotin; Enlimomab pegol; Enoblituzumab; Enokizumab; Enoticumab; Ensituximab; Epitumomab cituxetan; Epratuzumab; Erlizumab; Ertumaxomab; Etanercept; Etaracizumab; Etrolizumab; Evinacumab; Evolocumab; Exbivirumab; Fanolesomab; Faralimomab; Farletuzumab; Fasinumab; Felvizumab; Fezakinumab; Ficlatuzumab; Figitumumab; Firivumab; Flanvotumab; Fletikumab; Fontolizumab; Foralumab; Foravirumab; Fresolimumab; Fulranumab; Futuximab; Galiximab; Ganitumab; Gantenerumab; Gavilimomab; Gemtuzumab ozogamicin; Gevokizumab; Girentuximab; Glembatumumab vedotin; Golimumab; Gomiliximab; Guselkumab; Ibalizumab; Ibritumomab tiuxetan; Icrucumab; Idarucizumab; Igovomab; Imalumab; Imciromab; Imgatuzumab; Inclacumab; Indatuximab ravtansine; Indusatumab vedotin; Infliximab; Inolimomab; Inotuzumab ozogamicin; Intetumumab; Ipilimumab; Iratumumab; Isatuximab; Itolizumab; Ixekizumab; Keliximab; Labetuzumab; Lambrolizumab; Lampalizumab; Lebrikizumab; Lemalesomab; Lenzilumab; Lerdelimumab; Lexatumumab; Libivirumab; Lifastuzumab vedotin; Ligelizumab; Lilotomab satetraxetan; Lintuzumab; Lirilumab; Lodelcizumab; Lokivetmab; Lorvotuzumab mertansine; Lucatumumab; Lulizumab pegol; Lumiliximab; Lumretuzumab; Mapatumumab; Margetuximab; Maslimomab; Matuzumab; Mavrilimumab; Mepolizumab; Metelimumab; Milatuzumab; Minretumomab; Mirvetuximab soravtansine; Mitumomab; Mogamulizumab; Morolimumab; Motavizumab; Moxetumomab pasudotox; Muromonab-CD3; Nacolomab tafenatox; Namilumab; Naptumomab estafenatox; Narnatumab; Natalizumab; Nebacumab; Necitumumab; Nemolizumab; Nerelimomab; Nesvacumab; Nimotuzumab; Nivolumab; Nofetumomab merpentan; Obiltoxaximab; Obinutuzumab; Ocaratuzumab; Ocrelizumab; Odulimomab; Ofatumumab; Olaratumab; Olokizumab; Omalizumab; Onartuzumab; Ontuxizumab; Opicinumab; Oportuzumab monatox; Oregovomab; Orticumab; Otelixizumab; Otlertuzumab; Oxelumab; Ozanezumab; Ozoralizumab; Pagibaximab; Palivizumab; Panitumumab; Pankomab; Panobacumab; Parsatuzumab; Pascolizumab; Pasotuxizumab; Pateclizumab; Patritumab; Pembrolizumab; Pemtumomab; Perakizumab; Pertuzumab; Pexelizumab; Pidilizumab; Pinatuzumab vedotin; Pintumomab; Placulumab; Polatuzumab vedotin; Ponezumab; Priliximab; Pritoxaximab; Pritumumab; Quilizumab; Racotumomab; Radretumab; Rafivirumab; Ralpancizumab; Ramucirumab; Ranibizumab; Raxibacumab; Refanezumab; Regavirumab; Reslizumab; Rilotumumab; Rinucumab; Rituximab; Robatumumab; Roledumab; Romosozumab; Rontalizumab; Rovelizumab; Ruplizumab; Sacituzumab govitecan; Samalizumab; Sarilumab; Satumomab pendetide; Secukinumab; Seribantumab; Setoxaximab; Sevirumab; Sibrotuzumab; Sifalimumab; Siltuximab; Simtuzumab; Siplizumab; Sirukumab; Sofituzumab vedotin; Solanezumab; Solitomab; Sonepcizumab; Sontuzumab; Stamulumab; Sulesomab; Suvizumab; Tabalumab; Tacatuzumab tetraxetan; Tadocizumab; Talizumab; Tanezumab; Taplitumomab paptox; Tarextumab; Tefibazumab; Telimomab aritox; Tenatumomab; Teneliximab; Teplizumab; Teprotumumab; Tesidolumab; Tetulomab; Ticilimumab; Tigatuzumab; Tildrakizumab; Tocilizumab; Toralizumab; Tosatoxumab; Tositumomab; Tovetumab; Tralokinumab; Trastuzumab; Tregalizumab; Tremelimumab; Trevogrumab; Tucotuzumab celmoleukin; Tuvirumab; Ublituximab; Ulocuplumab; Urelumab; Urtoxazumab; Ustekinumab; Vandortuzumab vedotin; Vantictumab; Vanucizumab; Vapaliximab; Varlilumab; Vatelizumab; Vedolizumab; Veltuzumab; Vepalimomab; Vesencumab; Visilizumab; Volociximab; Vorsetuzumab mafodotin; Votumumab; Zalutumumab; Zanolimumab; Zatuximab; Ziralimumab; Zolimomab aritox.

In one example, the compound(s) of the disclosure is used in combination with at least one additional known compound which is currently being used or is in development for preventing or treating a chronic kidney disease. Examples of such compounds include but are not limited to: ACE inhibitor drugs (e.g. captopril (Capoten™), enalapril (Innovace™), fosinopril (Staril™), lisinopril (Zestril™) perindopril (Coversyl™), quinapril (Accupro™), trandanalopril (Gopten™), lotensin, moexipril, ramipril); RAS blockers; angiotensin receptor blockers (ARBs) (e.g. Olmesartan, Irbesartan, Losartan, Valsartan, candesartan, eprosartan, telmisartan, etc); protein kinase C (PKC) inhibitors (e.g. ruboxistaurin); inhibitors of AGE-dependent pathways (e.g. aminoguanidine, ALT-946, pyrodoxamine (pyrododorin), OPB-9295, alagebrium); anti-inflammatory agents (e.g. clyclooxigenase-2 inhibitors, mycophenolate mophetil, mizoribine, pentoxifylline), GAGs (e.g. sulodexide (U.S. Pat. No. 5,496,807)); pyridoxamine (U.S. Pat. No. 7,030,146); endothelin antagonists (e.g. SPP 301), COX-2 inhibitors, PPAR-gamma antagonists and other compounds like amifostine (used for cisplatin nephropathy), captopril (used for diabetic nephropathy), cyclophosphamide (used for idiopathic membranous nephropathy), sodium thiosulfate (used for cisplatin nephropathy).

In one example, the compound(s) of the disclosure is used in combination with at least one additional known compound which is currently being used or is in development for preventing or treating diabetes. Examples of such known compounds include but are not limited to common anti-diabetic drugs such as sulphonylureas (e.g. glicazide, glipizide), metformin, glitazones (e.g. rosiglitazone, pioglitazone), prandial glucose releasing agents (e.g. repaglinide, nateglinide), acarbose and insulin (including all naturally-occurring, synthetic and modified forms of insulin, such as insulin of human, bovine or porcine origin; insulin suspended in, for example, isophane or zinc and derivatives such as insulin glulisine, insulin lispro, insulin lispro protamine, insulin glargine, insulin detemir or insulin aspart).

As will be apparent from the foregoing, the present disclosure provides methods of concomitant therapeutic treatment of a subject, comprising administering to a subject in need thereof an effective amount of a first compound and a second compound, wherein said agent is a compound of the disclosure (i.e., an inhibitor of VEGF-B signaling), and the second agent is for the prevention or treatment of cancer, chronic kidney disease and/or diabetes.

As used herein, the term “concomitant” as in the phrase “concomitant treatment” includes administering a first agent in the presence of a second agent. A concomitant therapeutic treatment method includes methods in which the first, second, third or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and as a second actor may administer to the subject a second agent and the administering steps may be executed at the same time, or nearly the same time, or at distant times, so long as the first agent (and/or additional agents) are after administration in the presence of the second agent (and/or additional agents). The actor and the subject may be the same entity (e.g., a human).

In one example, the disclosure also provides a method for treating a wasting disorder in a subject, the method comprising administering to the subject a first pharmaceutical composition comprising at least one compound of the disclosure and a second pharmaceutical composition comprising one or more additional compounds.

In one example, a method of the disclosure comprises administering an inhibitor of VEGF-B signaling to a subject suffering from cachexia (e.g., cancer cachexia, CKD cachexia or diabetic cachexia) and receiving another treatment (e.g., for cancer, CKD or diabetes).

### Kits

Another example of the disclosure provides kits containing compounds useful for the treatment of stroke as described above.

In one example, the kit comprises (a) a container comprising a compound that inhibits VEGF-B signaling as described herein and/or an additional therapeutic compound as described herein, optionally in a pharmaceutically acceptable carrier or diluent; and (b) a package insert with instructions for treating a wasting disorder in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating the stroke and may have a sterile access port (for example, the container may be an intravenous solution bag or a vial having a stopper pierceable by a hypodermic injection needle). At least one active agent in the composition is the compound that inhibits VEGF-B signaling. The label or package insert indicates that the composition is used for treating a subject eligible for treatment, e.g., one having a wasting disorder, with specific guidance regarding dosing amounts and intervals of compound and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The present disclosure includes the following non-limiting Examples.

### EXAMPLES

### Example 1: Mice Deficient in VEGF-B have Reduced Basal Lipolysis Rate

Mice Deficient in VEGF-B are Heavier, but have Reduced Body Weight Loss During Fasting

Male C57BL/6 wild-type mice and aged matched (12 weeks) Vegfb^{+/-} and Vegfb^{-/-} mice were subjected to 14 hours of overnight fasting. Body weight and blood glucose levels were recorded before and after fasting. Body weight drop was calculated as a percentage of weight loss determined by recording body weight in the fasted and fed state. Glucose measurements were performed using a Bayer Contour Glucose meter on blood from the tail vein. Statistical analyses were performed using one-way ANOVA.

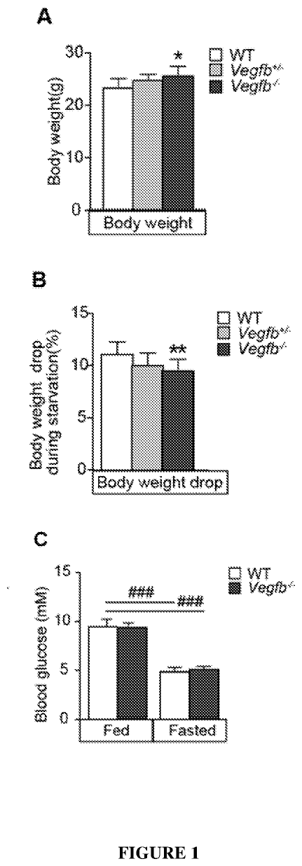

As shown in FIG. 1, chow-fed Vegfb^{-/-} mice show a small increase in body weight compared to wild-type littermates. Vegfb^{-/-} mice have reduced body weight loss during fasting, as compared to wild-type littermates.

These data show that ablation of Vegfb did not alter blood glucose levels, neither in the fed nor fasted state.

### Deletion of Vegfb in C57/BL6 Mice Prevents Increased Levels of Plasma Non-Esterified Fatty Acids and Glycerol in a Non-Insulin Dependent Manner, During Fasting-Induced Lipolysis

Male C57BL/6 mice and aged matched Vegfb^{+/-} and Vegfb^{-/-} (12 weeks old) mice were subjected to 14 hours of overnight fasting. Animals were sacrificed using carbon dioxide anaesthetics and total blood was removed by cardiac puncture. Blood was centrifuged at 14000 rpm, 4° C. for 10 minutes, serum was separated and frozen in aliquots at −80° C. Commercially available kits were used for enzymatic determination of non-esterified fatty acids (NEFAs; Wako Chemicals), glycerol (Abcam), triglycerides (TAGs; Sigma-Aldrich) and insulin (Mercodia). Statistical analyses were performed using one-way ANOVA.

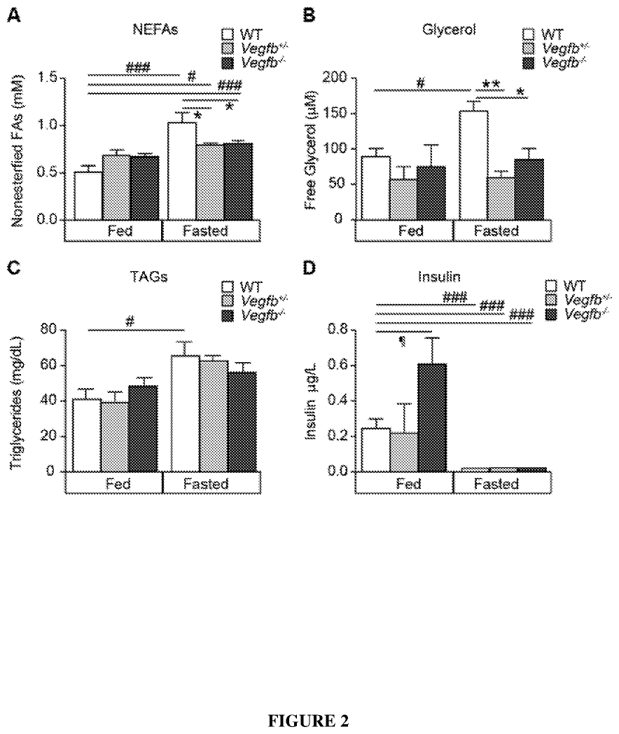

As shown in FIG. 2, deletion of Vegfb in chow-fed mice did not alter plasma levels of NEFAs, glycerol or TAGs, but a significant increase in plasma insulin levels was observed in Vegfb^{-/-} mice. In contrast during fasting, the release of NEFAs and glycerol and TAGs was significantly reduced in Vegfb^{+/-} and Vegfb^{-/-} mice. No difference in fasting plasma TAG or insulin levels were found between genotypes.

These data show that reduced levels of plasma NEFAs and glycerol were not caused by increased insulin secretion and insulin-induced suppression of lipolysis in Vegfb^{+/-} and Vegfb^{-/-} mice during fasting.

Basal Lipolysis Rate is Decreased in C57/BL6 Mice with Ablated Vegfb Expression

Male C57BL/6 wild-type mice and aged matched Vegfb^{-/-} mice (12 weeks old) were subjected to 14 hours of overnight fasting. Animals were sacrificed using carbon dioxide anaesthetics and visceral epididymal adipose tissue was surgically removed, washed in DPBS, and incubated in prewarmed (37° C.) DMEM (Dulbecco's modified Eagle's medium (DMEM, 1 g/l glucose; GIBCO, Life Technologies, Carlsbad, Calif.) until use. For measurements of forskolin-stimulated lipolysis, the adipose tissue pieces (20±5 mg) were pre-incubated in 200 μl DMEM containing 2% BSA (FA-free; Sigma-Aldrich) or 10 μM forskolin (Sigma-Aldrich) in the presence or absence of or 5 μM Atglistatin (Sigma-Aldrich) in 96-well plates at 37° C., 5% CO2, and 95% humidified atmosphere for 60 min. For measurements of basal lipolysis no pre-incubation was performed. Adipose tissue explants were transferred into 200 μl of identical fresh medium and incubated for further 60 min (=period of measurement) at 37° C., 5% CO2, and 95% humidified atmosphere. FA content was determined from the incubation media using a NEFA kit (Wako chemicals). The total amount of protein was measured by transferring the tissue explants into 1 ml extraction solution (Chloroform/methanol (2:1, v/v), 1% glacial acetic acid) and incubated for 60 min at 37° C. under vigorous shaking. The adipose tissue explants were then transferred to 500 μl lysis solution (NaOH/SDS (0.3 N/0.1%)) and incubated overnight at 55° C. under vigorous shaking. Protein content of the adipose tissue explant lysates was determined from the lysed solution using BCA reagent kit (Pierce) and BSA as standard. The rate of lipolysis rate was calculated as the amount of NEFAs/mg protein/hrs. Statistical analyses were performed using one-way ANOVA.

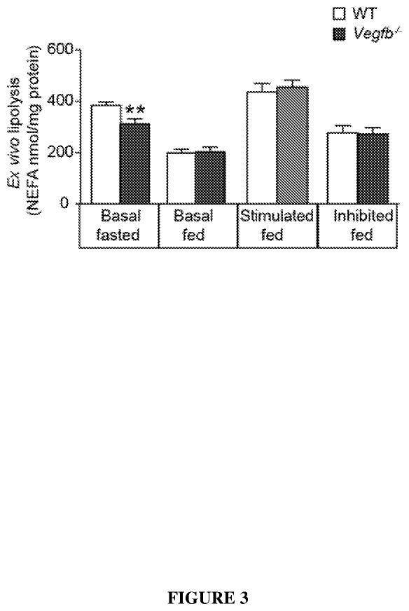

As shown in FIG. 3, lipolysis was readily induced by overnight fasting of wild-type mice. Basal lipolysis rate was significantly reduced in Vegfb^{-/-} mice, as compared to wild-type mice. In the presence of forskolin to stimulate adenylate cyclase activity, no difference in lipolysis rate between genotypes was detected. In the presence of Atglistatin forskolin-stimulated lipolysis was decreased both in wild-type and Vegfb^{-/-} mice.

Upregulation of Atgl Expression is Blunted in C57/BL6 Mice with Ablation of Vegfb During Fasting-Induced Lipolysis

Male C57BL/6 mice and aged matched Vegfb^{+/-} and Vegfb^{-/-} (12 weeks old) mice were subjected to 14 hours of overnight fasting. Animals were sacrificed with carbon dioxide anaesthetics and visceral epididymal adipose tissue was dissected. For expressional analysis total RNA was extracted and purified from tissues using the RNeasy Mini kit (Qiagen) according to the manufacturer's instructions. First strand cDNA was synthesized from 0.5-1 μg total RNA using iScript cDNA Synthesis Kit (Bio-Rad). Real-Time quantitive PCR was performed using KAPA SYBR FAST qPCR Kit Master Mix (2×) Universal (KAPA Biosystems) in Rotor-Gene Q (Qiagen) Real-Time PCR thermal cycler according to the manufacturers' instructions. Expression levels of adipose triglyceride lipase (Atgl), a major transcriptionally regulated lipase of lipolysis, and hormone sensitive lipase (Lipe) were determined and normalized to the expression of L19 and β-2 microglobulin. Statistical analyses were performed using one-way ANOVA.

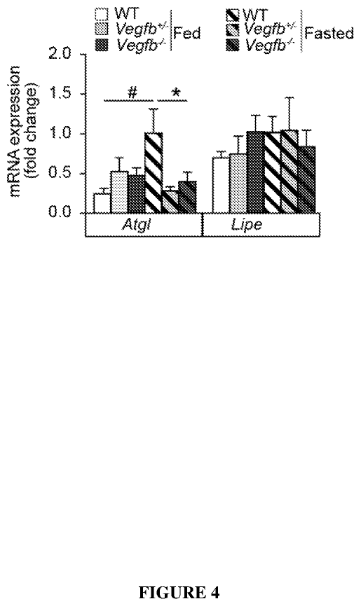

As shown in FIG. 4, in chow-fed mice Atgl was expressed independently of Vegfb expression in white adipose tissue (WAT). Fasting of wild-type mice readily activated WAT lipolysis and expression of Atgl was upregulated by 50%, as compared to chow-fed mice. In contrast, ablation of Vegfb normalised WAT Atgl levels in fasted mice. Expression of Lipe was not altered, neither during fasting of wild-type mice or in chow-fed or fasted Vegfb^{+/-} and Vegfb^{-/-} mice.

These data show that the decrease in basal lipolysis rate observed in fasted Vegfb^{-/-} results in a direct effect on Atgl by VEGF-B signalling in WAT.

Deletion of Vegfb in C57/BL6 Mice Reduces Hepatic Lipid Accumulation without Affecting Cardiac Lipid Uptake, During Fasting-Induced Lipolysis

Male C57BL/6 mice and aged matched (12 weeks) Vegfb^{+/-} and Vegfb^{-/-} mice were subjected to 14 hours of overnight fasting. Animals were sacrificed using carbon dioxide anaesthetics and livers and hearts were dissected. For Oil Red 0 (ORO) analysis, liver biopsies were embedded in Tissue-Tek® (Sakura) directly on the mould of the cryostat. Cryo sections (12 μm) were immersed either 5 min (liver) or 8 min (heart) in ORO working solution (2.5 g oil red 0 (Sigma-Aldrich), dissolved in 400 ml 99% isopropanol, further diluted 6:10 in H2O, filtered through a 22 μm filter (Corning). Thereafter the sections were submerged for 3 secs in hematoxylin solution followed by short submerging in LiCO₃ (for liver) and rinsed for 10 min under running tap water before they were mounted. Stained sections were examined with bright field microscopy. At least 10 frames per animal within each section were photographed with an Axio Vision microscope (Carl Zeiss) at 20× magnification. The amount of neutral lipids was quantified using Axio Vision Run wizard program for liver or heart ORO staining (pixel²/μm²). For measurements of lipid droplets, liver biopsies were fixed in 4% PFA for 24 hours and subsequently processed for paraffin embedding using standard procedures and 4 μm sections were prepared. Antigen retrieval was performed using Antigen retrieval solution Ph6 (Dako #S2367) and heating at 98° C. for 10 min. Sections were incubated at 4° C. overnight with guinea pig anti-adipophilin (Fitzgerald) antibody. Before addition of appropriate fluorescently labelled secondary antibody (Invitrogen, Alexa Fluor) samples were incubated with biotinylated donkey anti-guinea pig antibody (Jackson) for 1 hour at room temperature. At least 10 frames per animal within each section were photographed with an Axio Vision microscope (Carl Zeiss) at 20× magnification. The amount of lipid droplets was quantified using Axio Vision Run wizard program for hepatic adipophilin+staining (pixel²/μm² or arbitrary units).

Statistical Analyses were Performed Using One-Way ANOVA

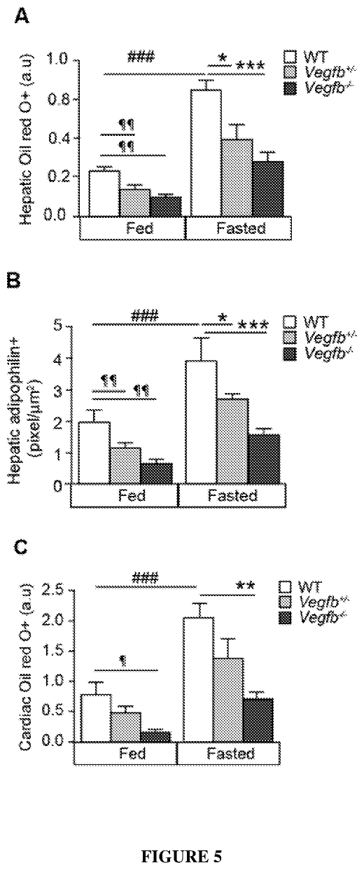

As shown in FIG. 5, hepatic lipid accumulation, measured by immune- or chemical based histological analyses, decreased by 30% in Vegfb^{+/-} and Vegfb^{-/-} chow-fed mice, as compared to wild-type chow-fed mice. Lipolysis induced hepatic lipid accumulation in wild-type mice, however this response was strongly decreased in mice with reduced Vegfb expression. This was not associated with increased accumulation of lipids in other peripheral tissues (i.e., heart) in Vegfb^{+/-} and Vegfb^{-/-} mice.

### Hepatic VEGF-B Expression and Signaling in C57/BL6 Mice is Low and not Induced During Fasting-Induced Lipolysis

Male C57BL/6 mice and aged matched Vegfb^{+/-} and Vegfb^{-/-} (12 weeks old) mice were subjected to 14 hours of overnight fasting. Animals were sacrificed using carbon dioxide anaesthetics and livers and quadriceps dissected. For expressional analysis total RNA was extracted and purified from tissues using the RNeasy Mini kit (Qiagen) according to the manufacturer's instructions. First strand cDNA was synthesized from 0.5-1 μg total RNA using iScript cDNA Synthesis Kit (Bio-Rad). Real-Time quantitive PCR was performed using KAPA SYBR FAST qPCR Kit Master Mix (2×) Universal (KAPA Biosystems) in Rotor-Gene Q (Qiagen) Real-Time PCR thermal cycler according to the manufacturer's instructions. Expression levels were normalized to the expression of L19 and β-2 microglobulin. Statistical analyses were performed using one-way ANOVA.

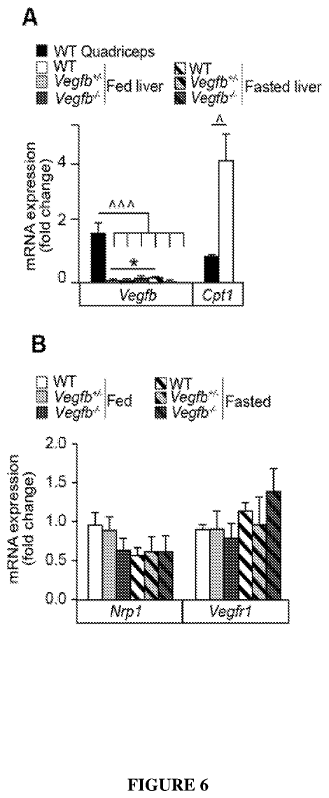

As shown in FIG. 6, in chow-fed mice, hepatic expression of Vegfb is minimal, close to the detection limit, and 56-folds lower as compared to Vegfb expression in wild-type quadriceps. A small increase in hepatic Vegfb transcript was observed during fasting, however it was still 38-fold lower as compared to wild-type quadriceps. No changes in hepatic expression of the VEGF-B receptors, Nrp-1 and VEGFR1 was observed, neither during fasting-induced lipolysis in wild-type mice or in mice with reduced expression of Vegfb.

These data show that that reducing VEGF-B signaling alters hepatic lipid accumulation via targeting WAT function such as e.g. fatty acid release, fatty acid storage or lipolysis.

### Expression of Hepatic Fatty Acid Transporters is Upregulated in Response to Fasting-Induced Lipolysis, Independently of Vegfb Expression

Male C57BL/6 mice and aged matched (12 weeks) Vegfb^{+/-} and Vegfb^{-/-} mice were subjected to 14 hours of overnight fasting. Animals were sacrificed using carbon dioxide anaesthetics and livers dissected. For expressional analysis total RNA was extracted and purified from livers using the RNeasy Mini kit (Qiagen) according to the manufacturer's instructions and cDNA synthesised as described above. Expression levels of Fatp1 (fatty acid transporter 1), Fatp2 (fatty acid transporter 2), Fatp4 (fatty acid transporter 4), Fatp5 (fatty acid transporter 5), CD36 (cluster of differentiation 36) and LPL (lipoprotein lipase) were measured and normalized to the expression of L19 and β-2 microglobulin. Statistical analyses were performed using two-way ANOVA

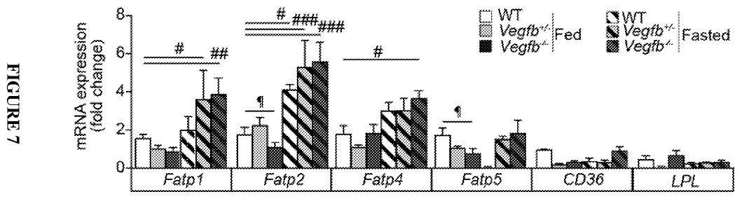

As shown in FIG. 7, in chow-fed conditions expression levels of Fatp2 and Fatp5, two of the major fatty acid (FA) transporters in the liver, were decreased in Vegfb^{-/-} mice. Fasting-induced lipolysis strongly upregulated hepatic expression of Fatp1, Fatp2 and Fatp4 up to 3-fold, compared to chow-fed wild-type animals.

These data show that deletion of Vegfb in mice subjected to fasting did not prevent up-regulation of transcript levels of hepatic Fatp1, Fatp2 and Fatp4. These data indicate that in fasting-induced lipolysis, hepatic Vegfb expression does not control FA uptake.

Lipolysis Rate is Decreased in C57/BL6 Mice with Specific Ablation of Vegfb in Adipocytes

Mice carrying the lox-containing gene construct allowing for Cre recombinase-mediated tissue specific ablation of Vegfb (Vegfb Flox+ mice) were generated by Taconic Artemis (Cologne, Germany). To generate mice with specific ablation of VEGF-B in the adipocytes, heterozygous mice carrying the Adiponectin promoter driven Cre recombinase expression cassette (AdiCre) were mated with heterozygous Vegfb flox+ mice. Male and female AdiCre/Vegfb Flox+, WT/WT, WT/Vegfb Flox+ and AdiCre/WT mice (12 weeks old) were subjected to 14 hours of overnight fasting. Animals were sacrificed using carbon dioxide anaesthetics and visceral epididymal adipose tissue was surgically removed, washed in DPBS, and incubated in pre-warmed (37° C.) DMEM (Dulbecco's modified Eagle's medium; 1 g/l glucose; GIBCO, Life Technologies, Carlsbad, Calif.) until use. For measurements of forskolin-stimulated lipolysis, the adipose tissue pieces (20±5 mg) were pre-incubated in 200 μl DMEM containing 2% BSA (FA-free; Sigma-Aldrich) or 10 μM forskolin (Sigma-Aldrich) in the presence or absence of or 5 μM Atglistatin (Sigma-Aldrich) in 96-well plates at 37° C., 5% CO2, and 95% humidified atmosphere for 60 min. For measurements of basal lipolysis no pre-incubation was performed. Adipose tissue explants were transferred into 200 μl of identical fresh medium and incubated for further 60 min (=period of measurement) at 37° C., 5% CO2, and 95% humidified atmosphere. FA content was determined from the incubation media using a NEFA kit (Wako chemicals). The total amount of protein was measured by transferring the tissue explants into 1 ml extraction solution (Chloroform/methanol (2:1, v/v), 1% glacial acetic acid) and incubated for 60 min at 37° C. under vigorous shaking. The adipose tissue explants were then transferred to 500 μl lysis solution (NaOH/SDS (0.3 N/0.1%)) and incubated overnight at 55° C. under vigorous shaking. Protein content of the adipose tissue explant lysates was determined from the lysed solution using BCA reagent kit (Pierce) and BSA as standard. The rate of lipolysis rate was calculated as the amount of NEFAs/mg protein/hrs. Statistical analyses were performed using one-way ANOVA.

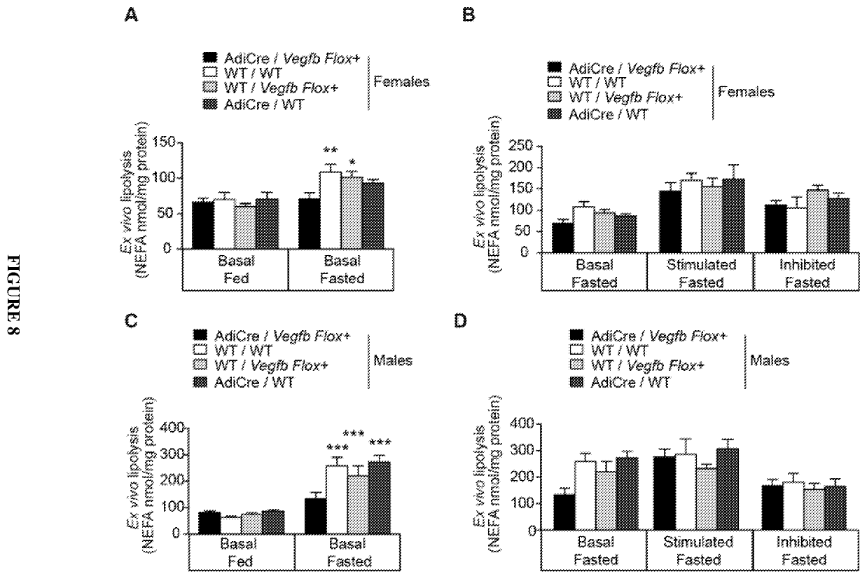

As shown in FIG. 8, lipolysis was readily induced by overnight fasting of WT/WT, WT/Vegfb Flox+ and AdiCre/WT but to a much lesser extent in AdiCre/Vegfb Flox+. Basal lipolysis rate was significantly reduced in both male and female AdiCre/Vegfb Flox+ mice as compared to all other relevant genotypes. In the presence of forskolin to stimulate adenylate cyclase activity, no difference in lipolysis rate between genotypes was detected. Atglistatin inhibited forskolin-stimulated lipolysis rate in all genotypes. These data showed that the VEGF-B mediated control of lipolysis is an adipocyte-autonomous effect.

### Example 2: A Neutralizing Anti-VEGF-B Antibody Targets Basal Lipolysis Rate

Anti-VEGF-B Treatment Using 2H10 in C56/BL6 Mice does not Influence Body Weight or Blood Glucose Levels, Neither in the Fed or Fasted State

C57BL/6 male mice purchased from Janvier labs and injected with 3 consecutive injections intraperitoneally (i.p.) twice weekly with 400 μg of anti-VEGF-B antibody (2H10) or an isotype matched control antibody. At the age of 12-16 weeks animals were subjected to 14 hours of overnight fasting. Body weight and blood glucose levels were determined as described above.

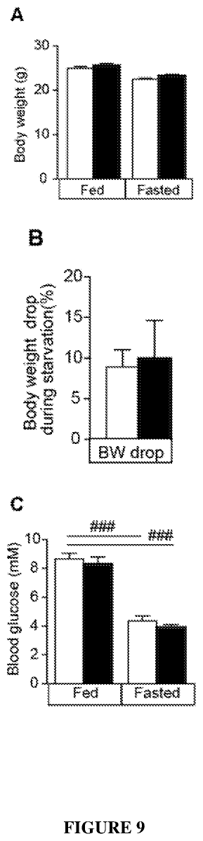

As shown in FIG. 9, reducing VEGF-B signalling in chow-fed or fasted mice did not impact on body weight or blood glucose levels.

### Anti-VEGF-B Treatment Using 2H10 in C57/B16 Mice Prevents Increased Levels of Plasma NEFAs and Glycerol in Fasting-Induced Lipolysis

C57BL/6 male mice were treated with an anti-VEGF-B antibody (2H10) or an isotype matched control antibody as described above. At the age of 12-16 weeks animals were subjected to 14 hours of overnight fasting, sacrificed using carbon dioxide anaesthetics and total blood removed by cardiac puncture and processed as described above. Enzymatic determination of NEFAs (Wako Chemicals) and glycerol (Abcam), triglycerides (Sigma-Aldrich) and insulin (Mercodia) was performed as previously described.

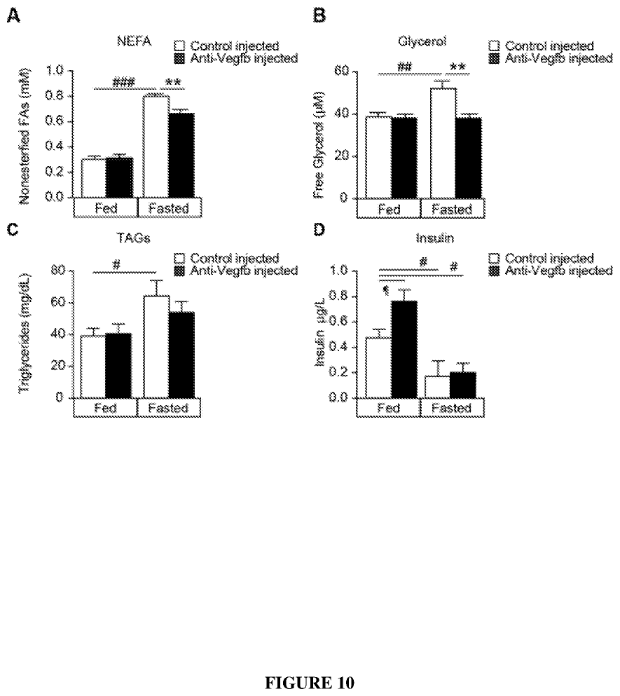

As shown in FIG. 10, reducing VEGF-B levels using antibody 2H10 in chow-fed mice did not alter plasma levels of NEFAs, glycerol or TAGs, but a significant increase in plasma insulin levels was observed in anti-VEGF-B treated mice. Fasting-induced lipolysis triggered the release of NEFAs and glycerol from WAT that increased the levels in the plasma in control treated mice. An increase in plasma TAG levels in control treated fasted mice was also observed. In contrast, plasma levels of NEFAs and glycerol were not increased in fasted mice treated with anti-VEGF-B antibody. No differences in fasting plasma TAG or insulin levels were found between different treatment groups.

These data show that reduced levels of plasma NEFAs and glycerol are not caused by increased insulin secretion and insulin-induced suppression of lipolysis in anti-VEGF-B treated mice during fasting.

### Anti-VEGF-B Treatment Using 2H10 in C56/BL6 Mice Targets Basal Lipolysis Rate

C57BL/6 male mice were treated with an anti-VEGF-B antibody (2H10) or an isotype matched control antibody as described above. At the age of 12-16 weeks animals were subjected to 14 hours of overnight fasting, sacrificed using carbon dioxide anaesthetics and visceral epididymal adipose tissue were surgically removed and processed as described above. Basal and forskolin-stimulated lipolysis was measured as previously described above.

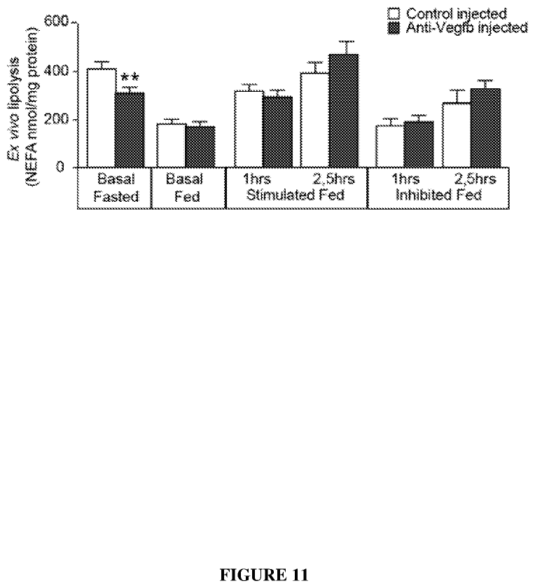

As shown in FIG. 11, lipolysis is readily induced by overnight fasting of control treated mice. Ex vivo lipolysis measurements shows that inhibition of VEGF-B signaling using anti-VEGF-B antibody 2H10 treatment significantly reduces basal lipolysis rate. This is in line with reduced basal lipolysis rate observed in fasted Vegfb^{-/-} mice. No effect on the forskolin-stimulated lipolysis rate was observed. In the presence of Atglistatin forskolin-stimulated lipolysis was decreased both in anti-VEGF-B and control treated C57BL/6 mice.

### Anti-VEGF-B Treatment Using 2H10 in C56/B16 Mice Prevents Hepatic Lipid Accumulation During Fasting-Induced Lipolysis

C57BL/6 male mice were treated with an anti-VEGF-B antibody (2H10) or an isotype matched control antibody as described above. At the age of 12-16 weeks animals were subjected to 14 hours of overnight fasting. Animals were sacrificed using carbon dioxide anaesthetics and livers dissected. Lipid droplet measurements in liver biopsies were performed as previously described above.

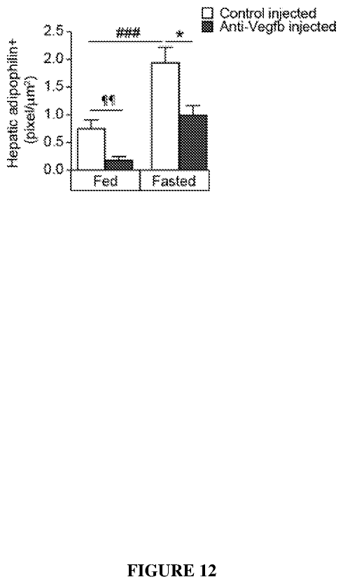

As shown in FIG. 12, fasting-induced lipolysis increased hepatic lipid accumulation in control treated mice. In contrast, anti-VEGF-B treatment significantly reduced hepatic lipid accumulation, both during fasting- and fed conditions.

These data show that anti-VEGF-B treatment using 2H10 inhibited hepatic lipid accumulation in response to fasting-induced lipolysis.

1. A method of treating a wasting disorder in a subject, the method comprising administering to the subject a compound that inhibits vascular endothelial growth factor B (VEGF-B) signaling.

2. The method of claim 1, wherein the subject is suffering from the wasting disorder.

3. The method of claim 1, wherein the wasting disorder is selected from the group consisting of cachexia, unintended body weight loss, fat wasting and anorexia.

4. The method of claim 3, wherein the cachexia is selected from the group consisting of cancer cachexia, chronic kidney disease cachexia and diabetic cachexia.

5. The method of claim 1, wherein the subject is suffering from cachexia.

6. A method of treating cancer cachexia in a subject suffering from cancer cachexia, the method comprising administering to the subject a compound that inhibits VEGF-B signaling.

7. The method of claim 1, wherein the compound is administered in an amount effective to have one or more of the following effects:
(a) reduce or prevent lipolysis;
(b) reduce or prevent hepatic lipid accumulation;
(c) reduce or prevent an increase in plasma non-esterified fatty acid levels; and/or
(d) reduce or prevent an increase in plasma free glycerol levels.

8. The method of claim 1, wherein the compound that inhibits VEGF-B signaling binds to VEGF-B.

9. The method of claim 8, wherein the compound is a protein comprising an antibody variable region that binds to or specifically binds to VEGF-B and neutralizes VEGF-B signaling.

10. The method of claim 9, wherein the compound is a protein comprising a fragment variable (Fv).

11. The method of claim 10, wherein the protein is selected from the group consisting of:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)2;
(viii) a Fv;
(ix) one of (i) to (ix) linked to a constant region of an antibody, a constant fragment (Fc) or a heavy chain constant domain (C_{H})2 and/or C_{H}3; and
(x) an antibody.

12. The method of claim 11, wherein the compound is a protein comprising an antibody variable region that competitively inhibits the binding of antibody 2H10 (comprising a heavy chain variable region (V_{H}) comprising a sequence set forth in SEQ ID NO: 3 and a light chain variable region (V_{L}) comprising a sequence set forth in SEQ ID NO: 4) to VEGF-B.

13. The method of claim 12, wherein the compound is a protein comprising a humanized form of a variable region of antibody 2H10 or the compound is a humanized form of antibody 2H10.

14. The method of claim 13, wherein the compound is an antibody comprising a VH comprising a sequence set forth in SEQ ID NO: 5 and a VL comprising a sequence set forth in SEQ ID NO: 6.

15. The method of claim 1, wherein the compound is a nucleic acid that inhibits VEGF-B signaling inhibits or prevents expression of VEGF-B.

16. The method of claim 15, wherein the nucleic acid is selected from the group an antisense, a siRNA, a RNAi, a ribozyme and a DNAzyme.

17. The method of claim 4, additionally comprising administering a further compound to treat the wasting disorder or to treat or prevent (or delay progression of) cancer, chronic kidney disease and/or diabetes.

18. The method of claim 12, wherein the compound comprises a variable region comprising the complementarity determining regions (CDRs) of the V_{H} and/or the V_{L} of antibody 2H10.

19. The method of claim 18, wherein the compound is an antibody or antigen binding fragment thereof comprising:
(i) a V_{H} comprising:
(a) a CDR1 comprising a sequence set forth in amino acids 25-34 of SEQ ID NO: 3;
(b) a CDR2 comprising a sequence set forth in amino acids 49-65 of SEQ ID NO: 3; and
(c) a CDR3 comprising a sequence set forth in amino acids 98-108 of SEQ ID NO: 3; and
(ii) a V_{L} comprising:
(a) a CDR1 comprising a sequence set forth in amino acids 23-33 of SEQ ID NO: 4;
(b) a CDR2 comprising a sequence set forth in amino acids 49-55 of SEQ ID NO: 4; and
(c) a CDR3 comprising a sequence set forth in amino acids 88-96 of SEQ ID NO: 4.

20. The method of claim 18, wherein the compound is an antibody or antigen binding fragment thereof comprising:
(i) a V_{H} comprising
(a) a CDR1 comprising a sequence set forth in SEQ ID NO: 20;
(b) a CDR2 comprising a sequence set forth in SEQ ID NO: 21; and
(c) a CDR3 comprising a sequence set forth in SEQ ID NO: 22; and
(ii) a V_{L} comprising:
(a) a CDR1 comprising a sequence set forth in SEQ ID NO: 17;
(b) a CDR2 comprising a sequence set forth in SEQ ID NO: 18; and
(c) a CDR3 comprising a sequence set forth in SEQ ID NO: 19.


SEQUENCE LISTING (46 sequences)

SEQ ID NO: 1 - protein, 207 aa, Homo sapiens
  SIGNAL (1)..(21): Signal sequence
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 2 - protein, 188 aa, Homo sapiens [sequence omitted]

SEQ ID NO: 3 - protein, 119 aa, artificial
  amino acid sequence from a VH of antibody 2H10
VQLQQPGTELVKPGASVKLSCKASGYTFTGFWIHWVKQRPGQGLEWIGHINPGNGGTNYNEKFKRMATLTVDKSSSTAYMQLSSLTSEDSAVYYCARSYSNYVRAMDYWGQGTSVTVSS

SEQ ID NO: 4 - protein, 106 aa, artificial
  amino acid sequence from a VL of antibody 2H10
IQMTQTTSSLSASLGDRVTISCRASQDISNFLNWYQQKPDGTVKLLIYYTSTLHSGVPSRFSGSGSGTDYSLTISNLEQEDIATYFCQQGKTLPPTFGGGTKLEIK

SEQ ID NO: 5 - protein, 119 aa, artificial
  amino acid sequence from a VH of a humanized form of antibody 2H10
VQLVQSGAEVKKPGASVKVSCKASGYTFTGFWIHWVRQAPGQGLEWMGHINPGNGGTNYNEKFKRRVTMTRDKSISTAYMELSRLRSDDTAVYYCARSYSNYVRAMDYWGQGTLVTVSS

SEQ ID NO: 6 - protein, 107 aa, artificial
  amino acid sequence of a VL of a humanized form of antibody 2H10
DIQMTQSPSSLSASVGDRVTITCRASQDISNFLNWYQQKPGKAPKLLIYYTSTLHSGVPSRFSGSGSGTDFTLTISSLQPEDFATYYCQQGKTLPPTFGGGTKVEIK

SEQ ID NO: 7 - protein, 116 aa, artificial
  amino acid sequence from a VH of antibody 4E12
VQPQQPGAELVKPGASVKMSCKASGDTFTNSWIGWVTQRPGQGLEWIGDIFPGSGHTNYNEKFKNRATLTVDTSSSTAYMLLSSLTSDDSAVYYCVIENYAWFAYWGQGTLVTVSA

SEQ ID NO: 8 - protein, 107 aa, artificial
  amino acid sequence of a VL of antibody 4E12
DIVMTQSQKFMSSSVGDRVSVTCKASQNVNTNVAWYQQKQGQSPRPLIYSASSRCSGVPDRFTGSGFGTDFTLTISNVQSEDLAEYFCQQYHSFPLTFGAGAKLDLK

SEQ ID NO: 9 - protein, 118 aa, artificial
  amino acid sequence from a VH of antibody 2F5
VQLQQSGPELVKPGTSVRISCKASGYTFTTFYIHWVKQRPGQGLEWIGWFYPGNVNTNYNEKLKGKATLTADKSSSAAYLQLNSLTSEDSAVYFCTRSPYYGYVFDYWGQGTTLTVSS

SEQ ID NO: 10 - protein, 107 aa, artificial
  amino acid sequence of a VL of antibody 2F5
DIVMTQSHKFMSTSVGDRVSITCKASQDVGTAVAWYQQKPGQSPKLLIYWASTRHTGVPDRFTGSGSGTDFTLTISNVQSEDLADYFCQQYSSSLTFGAGATKLELK

SEQ ID NO: 11 - DNA, 33 bp, Artificial Sequence
  Nucleotide sequence of 2H10 VL CDR1
agggcaagtcaggacattagcaattttttaaac

SEQ ID NO: 12 - DNA, 21 bp, Artificial Sequence
  Nucleotide sequence of 2H10 VL CDR2
tacacatcaacattacactca

SEQ ID NO: 13 - DNA, 27 bp, Artificial Sequence
  Nucleotide sequence of 2H10 VL CDR3
caacagggtaaaacgcttcctcccacg

SEQ ID NO: 14 - DNA, 30 bp, Artificial Sequence
  Nucleotide sequence of 2H10 VH CDR1
ggctacactttcactggcttctggatacac

SEQ ID NO: 15 - DNA, 51 bp, Artificial Sequence
  Nucleotide sequence of 2H10 VH CDR2
catattaatcctggcaatggtggcactaactacaatgagaagttcaagaga

SEQ ID NO: 16 - DNA, 33 bp, Artificial Sequence
  Nucleotide sequence of 2H10 VH CDR3
tcctatagtaactacgtgcgggctatggactac

SEQ ID NO: 17 - protein, 11 aa, Artificial Sequence
  Amino acid sequence of 2H10 VL CDR1
RASQDISNFLN

SEQ ID NO: 18 - protein, 7 aa, Artificial Sequence
  amino acid sequence of 2H10 VL CDR2
YTSTLHS

SEQ ID NO: 19 - protein, 9 aa, Artificial Sequence
  amino acid sequence of 2H10 VL CDR3
QQGKTLPPT

SEQ ID NO: 20 - protein, 10 aa, Artificial Sequence
  amino acid sequence of 2H10 VH CDR1
GYTFTGFWIH

SEQ ID NO: 21 - protein, 17 aa, Artificial Sequence
  amino acid sequence of 2H10 VH CDR2
HINPGNGGTNYNEKFKR

SEQ ID NO: 22 - protein, 11 aa, Artificial Sequence
  amino acid sequence of 2H10 VH CDR3
SYSNYVRAMDY

SEQ ID NO: 23 - DNA, 33 bp, Artificial Sequence
  nucleotide sequence of 2F5 VL CDR1
aaggccagtcaggatgtgggtactgctgtagcc

SEQ ID NO: 24 - DNA, 21 bp, Artificial Sequence
  nucleotide sequence of 2F5 VL CDR2
tgggcatccacccggcacact

SEQ ID NO: 25 - DNA, 24 bp, Artificial Sequence
  nucleotide sequence of 2F5 VL CDR3
caacaatatagcagctctctcacg

SEQ ID NO: 26 - DNA, 30 bp, Artificial Sequence
  nucleotide sequence of 2F5 VH CDR1
ggctacaccttcacaaccttctatatacac

SEQ ID NO: 27 - DNA, 51 bp, Artificial Sequence
  nucleotide sequence of 2F5 VH CDR2
tggttttatcctggaaatgttaataccaactacaatgagaagctcaagggc

SEQ ID NO: 28 - DNA, 30 bp, Artificial Sequence
  nucleotide sequence of 2F5 VH CDR3
tccccttactacggctacgtttttgactac

SEQ ID NO: 29 - protein, 11 aa, Artificial Sequence
  amino acid sequence of 2F5 VL CDR1
KASQDVGTAVA

SEQ ID NO: 30 - protein, 7 aa, Artificial Sequence
  amino acid sequence of 2F5 VL CDR2
WASTRHT

SEQ ID NO: 31 - protein, 8 aa, Artificial Sequence
  amino acid sequence of 2F5 VL CDR3
QQYSSSLT

SEQ ID NO: 32 - protein, 10 aa, Artificial Sequence
  amino acid sequence of 2F5 VH CDR1
GYTFTTFYIH

SEQ ID NO: 33 - protein, 17 aa, Artificial Sequence
  amino acid sequence of 2F5 VH CDR2
WFYPGNVNTNYNEKLKG

SEQ ID NO: 34 - protein, 10 aa, Artificial Sequence
  amino acid sequence of 2F5 VH CDR3
SPYYGYVFDY

SEQ ID NO: 35 - DNA, 33 bp, Artificial Sequence
  nucleotide sequence of 4E12 VL CDR1
aaggccagtcagaatgtgaacactaatgtagcc

SEQ ID NO: 36 - DNA, 21 bp, Artificial Sequence
  nucleotide sequence of 4E12 VL CDR2
tcggcatcctcccggtgcagt

SEQ ID NO: 37 - DNA, 27 bp, Artificial Sequence
  nucleotide sequence of 4E12 VL CDR3
cagcaatatcacagctttccgctcacg

SEQ ID NO: 38 - DNA, 30 bp, Artificial Sequence
  nucleotide sequence of 4E12 VH CDR1
ggcgacaccttcaccaactcctggataggc

SEQ ID NO: 39 - DNA, 51 bp, Artificial Sequence
  nucleotide sequence of 4E12 VH CDR2
gatatttttcctgggagtggtcatactaactacaatgagaagttcaagaac

SEQ ID NO: 40 - DNA, 24 bp, Artificial Sequence
  nucleotide sequence of 4E12 VH CDR3
gagaattatgcctggtttgcttat

SEQ ID NO: 41 - protein, 11 aa, Artificial Sequence
  amino acid sequence of 4E12 VL CDR1
KASQNVNTNVA

SEQ ID NO: 42 - protein, 7 aa, Artificial Sequence
  amino acid sequence of 4E12 VL CDR2
SASSRCS

SEQ ID NO: 43 - protein, 9 aa, Artificial Sequence
  amino acid sequence of 4E12 VL CDR3
QQYHSFPLT

SEQ ID NO: 44 - protein, 10 aa, Artificial Sequence
  amino acid sequence of 4E12 VH CDR1
GDTFTNSWIG

SEQ ID NO: 45 - protein, 17 aa, Artificial Sequence
  amino acid sequence of 4E12 VH CDR2
DIFPGSGHTNYNEKFKN

SEQ ID NO: 46 - protein, 8 aa, Artificial Sequence
  amino acid sequence of 4E12 VH CDR3
ENYAWFAY